(19) United States
(12) Patent Application Publication
(10) Pub. No.: US 2019/0133687 A1
(43) Pub. Date: May 9, 2019

(54) FIBEROPTIC FOR MEDICAL APPLICATIONS

(71) Applicant: ASYMMETRIC MEDICAL LTD., Kfar Mordechai (IL)

(72) Inventors: Moshe ESHKOL, Harutzim (IL); Ori WEISBERG, Kfar Mordechai (IL)

(73) Assignee: ASYMMETRIC MEDICAL LTD., Kfar Mordechai (IL)

(21) Appl. No.: 16/094,514

(22) PCT Filed: Apr. 19, 2017
(86) PCT No.: PCT/IL17/50460

§ 371 Date: Oct. 18, 2018

Related U.S. Application Data
(60) Provisional application No. 62/324,378, filed on Apr. 19, 2016.
(60) Provisional application No. 62/419,231, filed on Nov. 8, 2016.

(51) Int. Cl. A61B 18/22; G02B 6/036; G02B 6/36

(52) U.S. Cl. CPC A61B 18/22; A61B 2018/00351; G02B 6/3624; G02B 6/036

(57) ABSTRACT

Medical treatment devices for treating a tissue are disclosed. The medical treatment devices may comprise an optical fiber with a treatment section as well as an illumination source configured to deliver electromagnetic radiation through the optical fiber to apply a specified treatment. The treatment section may comprise at least one element with a refractive index that may be different from a refractive index of the optical fiber and/or may be same as the refractive index of the optical fiber. A spatial configuration of the optical and the at least one element within the treatment section of the medical treatment device and/or optical properties of the optical fiber and/or the at least one element may determine an emission region through the treatment section. The emission region may be radial (or at least have a radial component) with respect to a cross-section of the optical fiber.

### CROSS REFERENCE TO RELATED APPLICATIONS

This application claims priority to U.S. Provisional Application No. 62/324,378 filed on Apr. 19, 2016 and to U.S. Provisional Application No. 62/419,213 filed on Nov. 8, 2016, which are incorporated herein by reference in their entirety.

### BACKGROUND OF THE INVENTION

### 1. Technical Field

The present invention relates to the field of optical fibers, and more particularly, to optical fibers configured to emit a delivered electromagnetic radiation laterally.

### 2. Discussion of Related Art

Current laser emitting medical treatment devices typically emit an electromagnetic radiation through a tip of an optical fiber.

### SUMMARY OF THE INVENTION

One aspect of the present invention provides a medical treatment device comprising: an optical fiber having a core refractive index n_{C} and a treatment section along a part of the length of the optical fiber; a cover attached to the optical fiber over the treatment section of the optical fiber; and at least one element having a refractive index n_{E} which is different from n_{C}, wherein the at least one element is embedded within the optical fiber over at least the treatment section; wherein a spatial configuration of the optical fiber within the treatment section and a spatial configuration of the at least one element within the treatment section are configured to determine an emission region of electromagnetic radiation from the optical fiber, the emission region being radial with respect to a cross section of the optical fiber and along the treatment section.

Another aspect of the present invention provides a medical treatment device, the device comprising: a supportive structure configured to position and to orient at least a portion of the device with respect to a predetermined target region; an optical fiber having a core refractive index n_{C} and a treatment section, wherein at least the treatment section is attached to the supportive structure; and wherein a spatial configuration of the optical fiber within the treatment section is configured to determine an emission region of electromagnetic radiation from the optical fiber to the predetermined target region, the emission region being along the treatment section.

Another aspect of the present invention provides a medical treatment device comprising: an optical fiber delivery unit; and a V-shape unit attached to the optical fiber delivery unit, the V-shape unit configured to emit electromagnetic radiation upon a target region.

Another aspect of the present invention provides a medical treatment device comprising: an optical fiber configured to emit electromagnetic radiation from a distal end of the optical fiber; a plurality of facets attached to the distal end of the optical fiber, the facets configured to distribute the emitted electromagnetic radiation in a curtain-like profile.

Another aspect of the present invention provides a medical treatment device comprising: a supportive structure; an optical fiber having a treatment section, wherein at least the treatment section is attached to the supportive structure, the treatment section having at least one emission region configured to emit electromagnetic radiation upon bending of the treatment section beyond a predetermined bending threshold.

Another aspect of the present invention provides a system for bending a treatment section of an optical fiber, the system comprising: an anchoring unit configured to removably attach the system to a work station; a first positioning element and a second positioning element removably attached to the anchoring unit and positioned at a predetermined distance from each other along a longitudinal axis of the system, the first positioning element and the second positioning element configured to receive and position the treatment section of the optical fiber in a predetermined location within the system; at least one heating element configured to elevate a temperature of the optical fiber, to a predetermined temperature threshold; and at least one bending element configured to shape the treatment section of the optical fiber into a predetermined shape.

Another aspect of the present invention provides a method comprising: inserting a treatment section of an optical fiber into an elongated glass member, heating the elongated glass member, forming a cover over the treatment section by pressing the heated elongated glass member to simultaneously cool and attach the glass member to the optical fiber, wherein the forming is carried out to bend the treatment section into a predefined shape, and configuring the inserting and the forming to provide a spatial configuration which defines an emission region of electromagnetic radiation from the optical fiber, the emission region being radial with respect to a cross section of the optical fiber and along the end section.

Another aspect of the present invention provides a method comprising: heating an optical fiber over a treatment section of a treatment device, bending the treatment section into a predefined shape to provide a spatial configuration which defines an emission region of electromagnetic radiation from the optical fiber, the emission region having a radial component with respect to a cross section of the optical fiber and along the end section.

These, additional, and/or other aspects and/or advantages of the present invention are set forth in the detailed description which follows; possibly inferable from the detailed description; and/or learnable by practice of the present invention.

### BRIEF DESCRIPTION OF THE DRAWINGS

For a better understanding of embodiments of the invention and to show how the same may be carried into effect, reference will now be made, purely by way of example, to the accompanying drawings in which like numerals designate corresponding elements or sections throughout.

In the accompanying drawings:

FIG. 1A is a high level schematic illustration of a medical treatment device having a treatment section, according to some embodiments of the invention;

FIGS. 1B-1C are high level schematic illustrations of a medical treatment device having a bended treatment section, according to some embodiments of the invention;

FIG. 1D is a high level schematic diagram illustrating the conditions for lateral emission in a curved region of treatment section of a medical treatment device, according to some embodiments of the invention;

FIGS. 2A-2E are high level schematic illustrations of a system for production of a medical treatment device with a treatment section, according to some embodiments of the invention;

FIGS. 3A-3M are high level schematic illustrations of various embodiments of treatment section of medical treatment device, according to some embodiments of the invention;

FIGS. 4A-4B are high level schematic illustrations of a medical treatment device having a treatment section coupled to a supportive structure and designed to emit electromagnetic radiation from convex portion of the treatment section upon a target region, according to some embodiments of the invention;

FIGS. 4C-4E are high level schematic illustrations of a medical treatment device having a treatment section coupled to a supportive structure and designed to emit electromagnetic radiation at a predetermined angle with respect to direction of bending upon a target region, according to some embodiments of the invention;

FIGS. 4F-4G are high level schematic illustrations of a medical treatment device with a treatment section having two optical fibers and two emission regions associated with the fibers, according to some embodiments of the invention;

FIGS. 4H-4K are high level schematic illustrations of various embodiments of supportive structures of medical treatment device, according to some embodiments of the invention;

FIGS. 5A-5B are high level schematic illustrations of a medical treatment device having a V-shape unit and optical fibers delivery unit, according to some embodiments of the invention;

FIGS. 5C-5D are high level schematic illustrations of a medical treatment device having at least one treatment section at a distal end of the device, according to some embodiments of the invention;

FIGS. 5E-5F are high level schematic illustrations of a chisel-like medical treatment device, according to some embodiments of the invention;

FIGS. 5G-5H are high level schematic illustrations of a hook-like medical treatment device, according to some embodiments of the invention;

FIG. 6 is a high level schematic flowchart of a method of fusing a glass member to an optical fiber over a treatment section of the optical fiber and bending thereof, according to some embodiments of the invention; and

FIG. 7 is a high level schematic flowchart of a method of bending a treatment section of an optical fiber, according to some embodiments of the invention.

### DETAILED DESCRIPTION OF THE INVENTION

In the following description, various aspects of the present invention are described. For purposes of explanation, specific configurations and details are set forth in order to provide a thorough understanding of the present invention. However, it will also be apparent to one skilled in the art that the present invention may be practiced without the specific details presented herein. Furthermore, well known features may have been omitted or simplified in order not to obscure the present invention. With specific reference to the drawings, it is stressed that the particulars shown are by way of example and for purposes of illustrative discussion of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

Before at least one embodiment of the invention is explained in detail, it is to be understood that the invention is not limited in its application to the details of construction and the arrangement of the components set forth in the following description or illustrated in the drawings. The invention is applicable to other embodiments that may be practiced or carried out in various ways as well as to combinations of the disclosed embodiments. Also, it is to be understood that the phraseology and terminology employed herein is for the purpose of description and should not be regarded as limiting.

Medical treatment devices for treating a tissue are disclosed. Medical treatment devices may comprise an optical fiber with at least one element embedded within the optical fiber over a treatment section as well as an illumination source configured to deliver electromagnetic radiation through the optical fiber to apply a specified treatment. Optionally, the medical treatment device may comprise a cover attached to the optical fiber over the treatment section. The treatment section may apply the specified treatment by emitting the delivered electromagnetic radiation laterally (e.g., sideways) along at least a part of its length, rather than through the fiber tip as is typical in current laser emitting devices. A spatial configuration of the optical fiber within the treatment section (e.g., bending of the treatment section beyond a predetermined bending threshold), a spatial configuration of the at least one element within the treatment section of the medical treatment device (e.g., number of elements, position of elements in a cross-section of the treatment section, a distance between adjacent elements, etc.) and/or optical properties (e.g., refractive indexes) of the optical fiber, the at least one element and optionally cover may determine an emission region in the treatment section. In some embodiments, the emission in the emission region may be radial (or at least have a radial component) with respect to a cross-section of the optical fiber. Accordingly, in some embodiments, at least some electromagnetic radiation may be emitted from the emission region laterally (e.g., sideways).

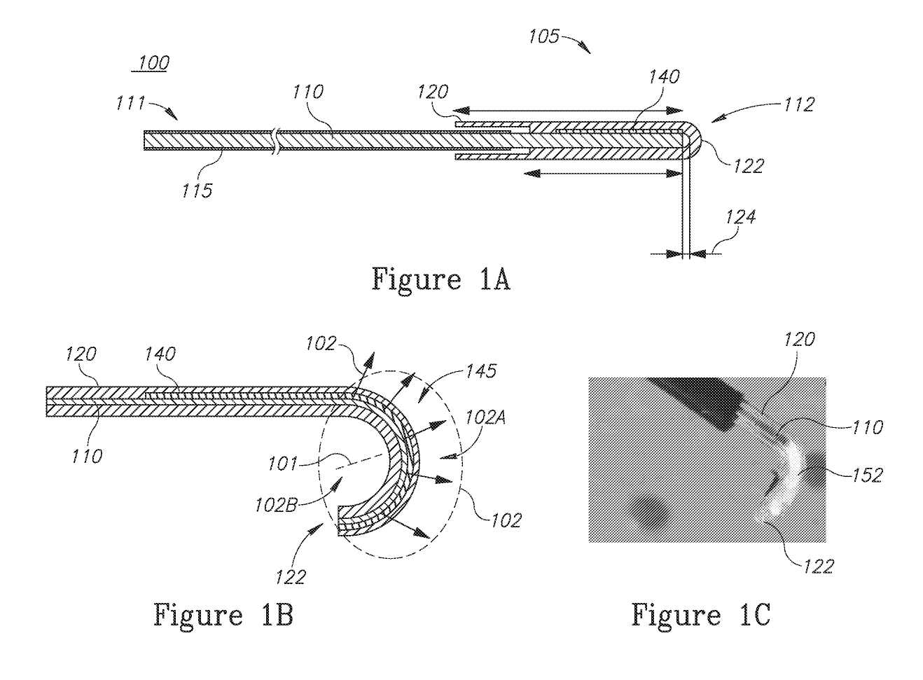

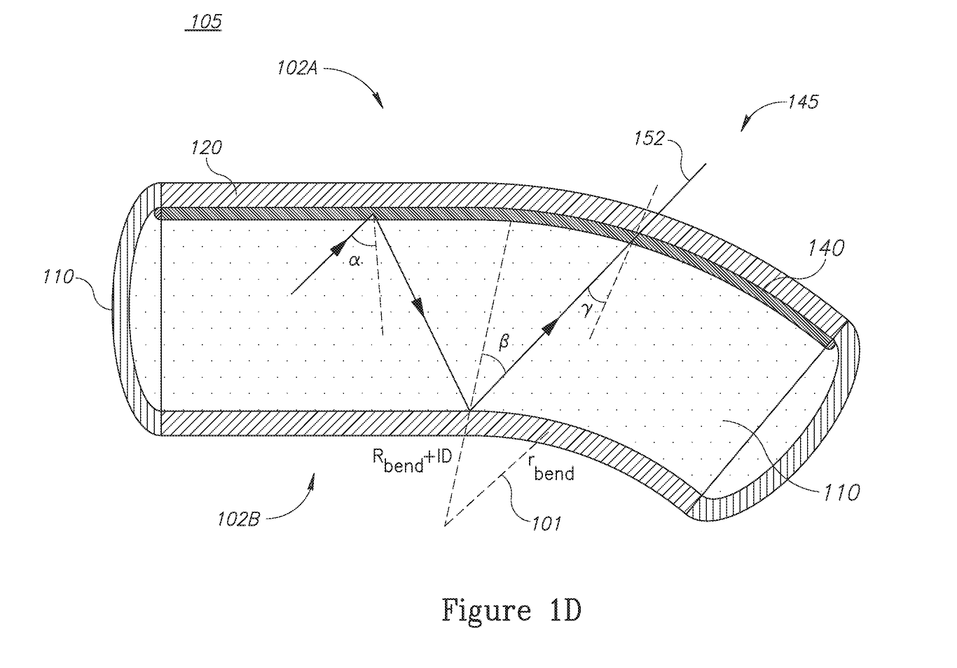

FIG. 1A is a high level schematic illustration of a medical treatment device 100 having a treatment section 105, according to some embodiments of the invention.

Medical treatment device 100 may comprise an optical fiber 110. Optical fiber 110 may be a radially and/or circumferentially symmetric fiber and/or may have a uniform refractive index profile n_{C}. In some embodiments, optical fiber 110 is asymmetric fiber. In some embodiments, optical fiber 110 has an internal structure such as, for example, a photonic-crystal (PCF)-like fiber, a microstructured fiber and/or OmniGuide fiber. At least a portion of optical fiber 110 may be coated with a coating 115. In various embodiments, optical fiber 110 is a structured fiber, a photonic-crystal fiber (PCF), a metallic wave-guide, an omniguide, a total internal reflection fiber, a core-less fiber and/or any other optical fiber and/or wave guide. In some embodiments, medical treatment device 100 may comprise a plurality of optical fibers 110.

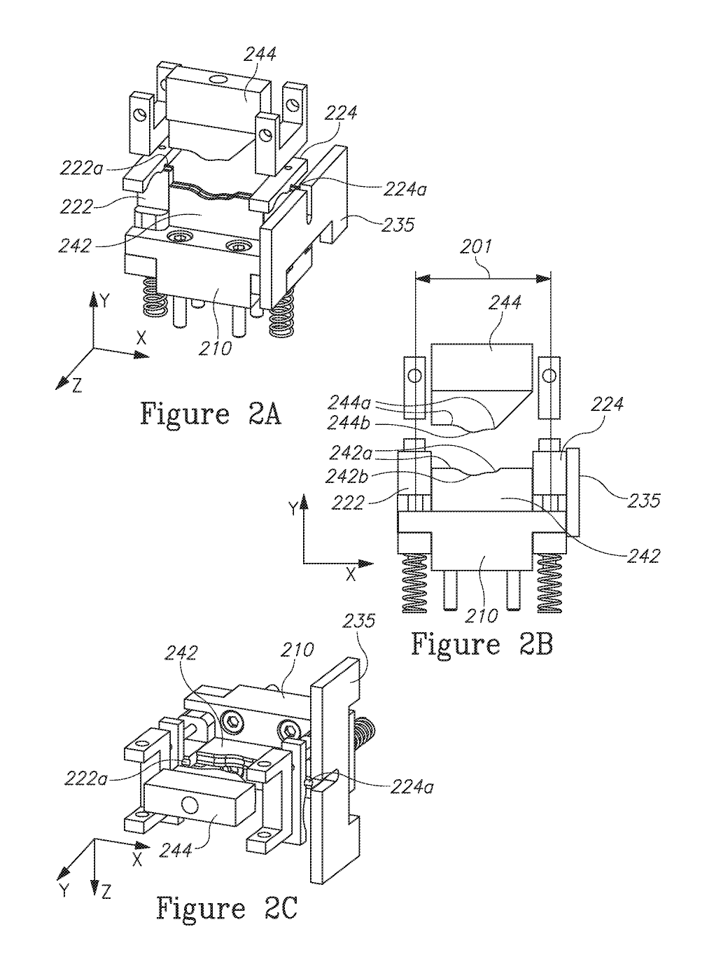

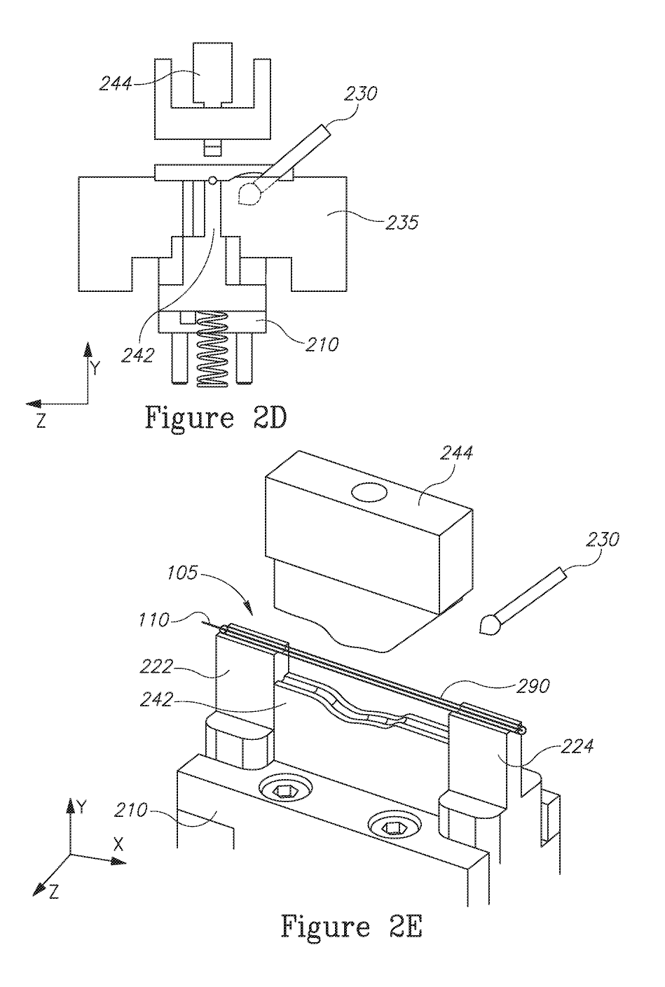

Optionally, medical treatment device 100 may comprise a cover 120 over a treatment section 105 that may be, for example, attached to optical fiber 110. In some embodiments, cover 120 is a protective cover configured to prevent a damage of optical fiber 110 in treatment section 105. Cover 120 may comprise a hollow cylindrical structure, such as a glass tube. Cover 120 may be fused with optical fiber 110 at treatment section 105, for example in a production process. In some embodiments, fusion of optical fiber 110 with cover 120 is done using a power source that may comprise, for example, a laser, an electrical heating and/or other heating means, as described in detail below with respect to FIGS. 2A-2E. In some embodiments, cover 120 is transparent to, for example, visible light. In some embodiments, cover 120 comprises at least one predetermined region which is transparent to electromagnetic radiation in a predetermined wavelength range. In some embodiments, cover 120 comprises at least one predetermined region which is opaque and/or reflective to electromagnetic radiation in a predetermined wavelength range.

In some embodiments, cover 120 is positioned at a distal end 112 of optical fiber 110. Cover 120 may comprise a cap 122 (e.g., as shown in FIG. 1A). Cap 122 may be positioned at a predetermined distance 124 from distal end 112 of optical fiber 110. Distance 124 may be predetermined to avoid reflection of electromagnetic radiation emitted from distal end 112 back to optical fiber 110. In various embodiments, cap 122 may comprise one or more beam-dump, reflector and/or anti-reflection optical element.

Medical treatment device 100 may comprise at least one element 140 having a refractive index n_{E}. For example, the refractive index n_{C} of optical fiber 110 and/or the refractive index n_{E} of element 140 may be in a 1.43-1.47 range (e.g., at a wavelength of 1 μm). Element 140 may comprise, for example, a doped silica glass, Ge, F and/or Al. In some embodiments, the refractive index n_{E} of element 140 is different from the refractive index n_{C} of optical fiber 110. For example, a normalized difference Δ between the refractive index n_{C} of optical fiber 110 and refractive index n_{E} of element 140 (e.g., as Δ=|(n_{C}−n_{E})/n_{C}|·100%) may be smaller than 1%. In some embodiments, the refractive index of n_{E} of element 140 is the same as the refractive index n_{C} of optical fiber 110.

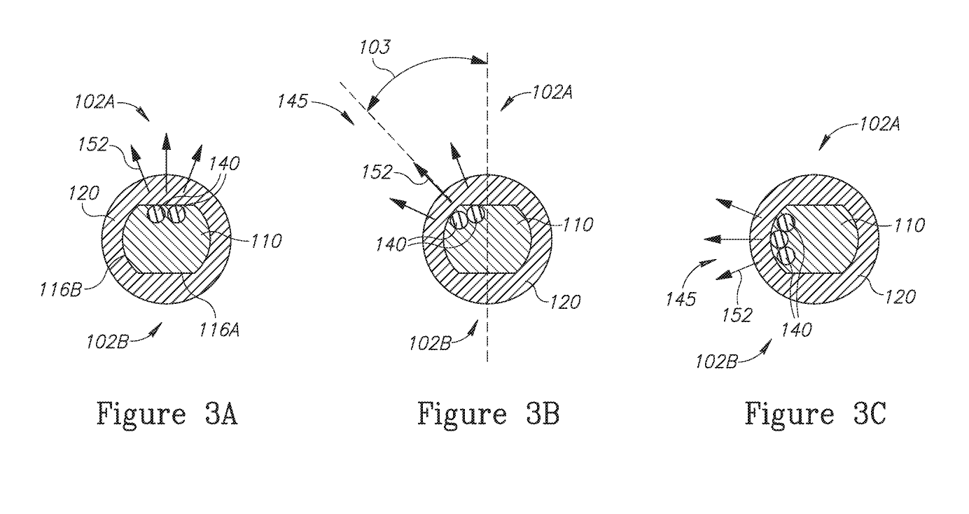

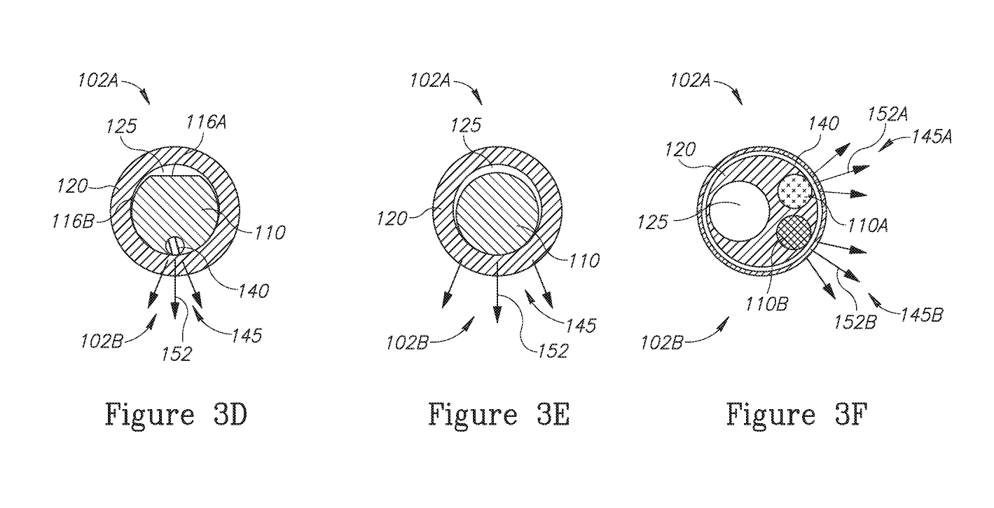

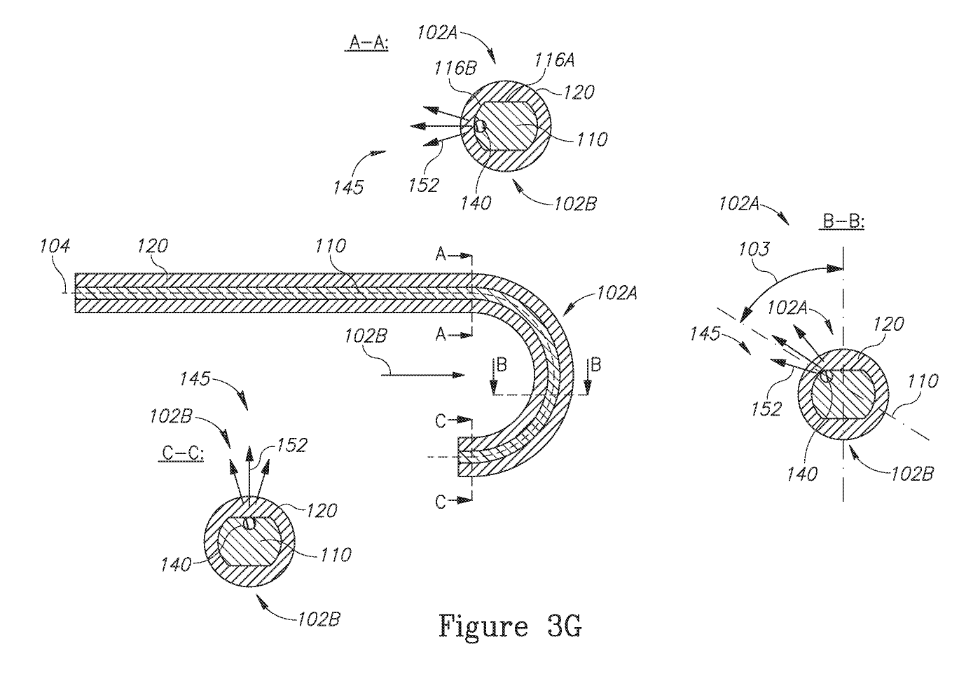

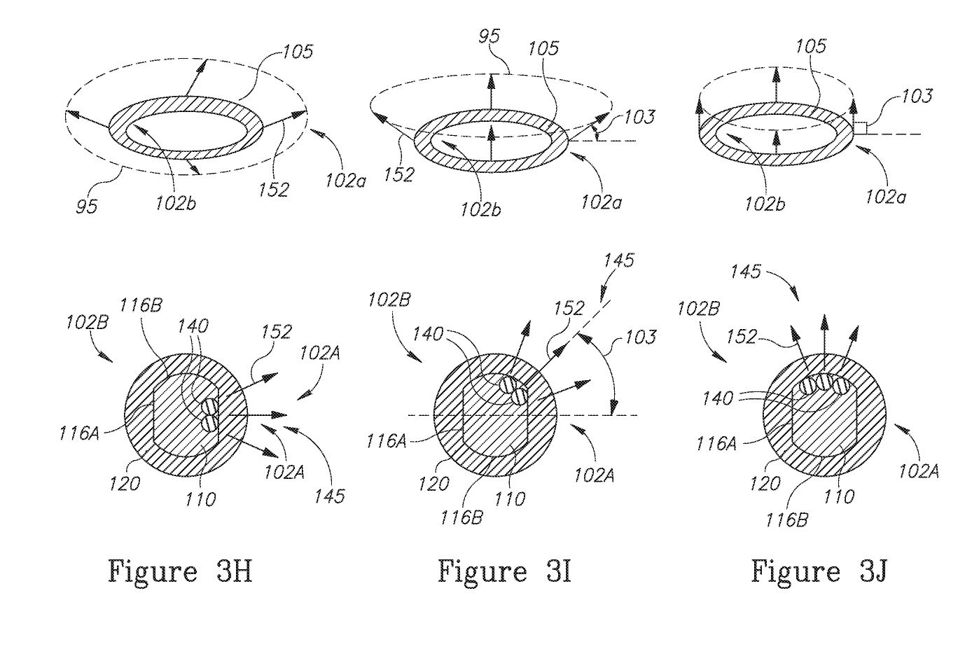

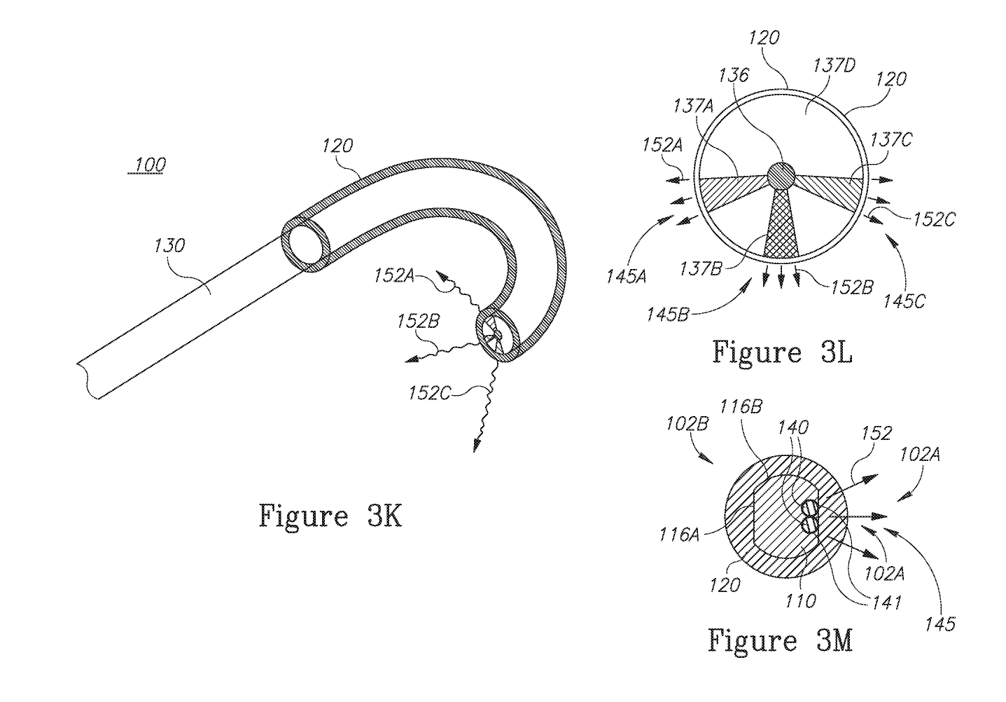

In various embodiments, element 140 is embedded within optical fiber 110 or optionally within cover 120 (e.g., in a production process, for example, as described in detail with respect to FIGS. 2A-2E). In some embodiments, element 140 is embedded at an interface between optical fiber 110 and cover 120. In various embodiments, element 140 is embedded within optical fiber 110 distant from optical fiber's 110 outer surface. Additionally or alternatively element 140 may comprise covering (e.g., additional to the element) that envelopes the element 140 (e.g., as shown in FIG. 3M). Production and/or embedding of element 140 within optical fiber 110 and/or cover 120 according to some embodiments of the invention is described below in detail with respect to FIGS. 2A-2E.

FIGS. 1B-1C are high level schematic illustrations of a medical treatment device 100 having a bent or curved treatment section 105, according to some embodiments of the invention. In some embodiments, at least treatment section 105 is inflexible, e.g. permanently curved, so that it retains its curved shape during normal use.

FIGS. 1B-1C show optical fiber 110 including an emission region 145 in treatment section 105 in which electromagnetic radiation may be emitted laterally from the fiber. For example, radiation may be emitted from the surface of the fiber in emission region 145 rather than being emitted from the end of the fiber, or tip. Emission region 145 may be defined in various ways including but not limited to: a spatial configuration of optical fiber 110 within treatment section 105 (e.g., bending of treatment section 105 beyond a predetermined bending threshold), a spatial configuration of elements 140 within treatment section 105 of medical treatment device 100 (e.g., number of elements 140, position of elements 140 in a cross-section of treatment section 105, a distance between adjacent elements 140, etc.) and/or optical properties (e.g., refractive indexes) of optical fiber 110, element 140 and optionally of cover 120. Emission region 145 may be radial with respect to a cross-section of optical fiber 110 and/or may be positioned along treatment section 105. Accordingly, the electromagnetic radiation may be emitted from emission region 145 laterally (e.g., sideways).

In some embodiments, medical treatment device 100 comprises one or more treatment sections 105 along the device (e.g., at a proximal end 111, at distal end 112 and/or any other location). In various embodiments, each of treatment sections 105 comprises at least one element 140 embedded in optical fiber 110 and/or cover 120 and/or at least one emission region 145. Each of emission regions 145 in each of treatment sections 105 may comprise different emitting characteristics.

In some embodiments, treatment section 105 may be shaped to provide a bent or curved region 102 (e.g., as shown in FIGS. 1B-1C) and/or may be done using a power source that may comprise, for example, a laser, an electrical heating and/or other heating means, as described below in detail with respect to FIGS. 2A-2E. Curved region 102 may comprise a convex and concave portions 102A, 102B, respectively (e.g., as shown in FIG. 1B). At least one of convex and/or concave portions 102A, 102B may comprise at least one emission region 145. Treatment section 105 may be shaped to various forms and/or structures that may comprise, for example, a mesh-like, a ring-like and/or hook-like form, depending on the application of medical treatment device 100.

In some embodiments, emission region 145 is defined (e.g., based on a spatial configuration of optical fiber 110 within treatment section 105, a spatial configuration of elements 140 within treatment section 105 of medical treatment device 100 and/or optical properties of optical fiber 110, element 140 and/or cover 120) to enable emission of a predetermined amount of electromagnetic radiation travelling along optical fiber 110 (e.g., at least 70%) through, for example, curved region 102 of treatment section 105.

FIG. 1D is a high level schematic diagram illustrating the conditions for lateral emission in a curved region 102 of treatment section 105 of a medical treatment device 100, according to some embodiments of the invention. FIG. 1D presents an example of possible trajectory of light travelling along treatment section 105.

A spatial configuration of optical fiber 110 (e.g., bending radius 101 of curved region 102), a position of element 140 (e.g., adjacent to convex portion 102A) and/or optical properties of optical fiber 110 and/or element 140 (e.g., refractive indexes n_{C}, n_{E}) may determine emission region 145. For example emission region 145 may be associated with element 140. In some embodiments, electromagnetic radiation reaches a convex portion 102A at the beginning of curved region 102 of treatment section 105 at an angle α that is larger than θₑₘᵢₛₛᵢₒₙ_{_}_{region}=sin⁻¹ (n_{E}/n_{C}).

The angle α may be not sufficient for the lateral emission due to bending radius that may be not sufficiently small at this location of treatment section 105. As a result, the light may be internally reflected at the interface between the fiber 110 and the cover 120 to a concave surface region 102B of the fiber 120. In some embodiments, the light is reflected from concave portion 102B at an angle β that is larger than θ_{protective}_{_}_{cover}=sin⁻¹ (n_{F}/n_{C}) and/or stays within optical fiber 110. In various embodiments, n_{F} is a refractive index of an air, a cladding surrounding a core of the optical fiber 110 (not shown) and/or of cover 120 (e.g., as shown FIG. 1D). In some embodiments, n_{F} is substantially lower as compared to n_{C}, n_{E}. In some embodiments, upon reflection from concave portion 102B, the light reaches convex portion 102A at an angle γ that may be smaller than θₑₘᵢₛₛᵢₒₙ_{_}_{region}=sin⁻¹ (n_{E}/n_{C}) due to bending radius 101 that may be beyond a predetermined bending threshold at this location of treatment section 105. Accordingly, the light may be emitted from emission region 145 (e.g., from outer curved region 102B, as shown in FIG. 1B).

Lateral emission may occur at around an angle θ_{bend} that may be based on bending radius 101 (e.g., r_{bend}), as follows: θ_{bend}=sin⁻¹ (r_{bend}/r_{bend}+ID)), where ID is an internal diameter of optical fiber 110. The condition for lateral emission may be as follows: sin⁻¹ (n_{F}/n_{C})=θ_{protective}_{_}_{cover}<θ_{bend}<θₑₘᵢₛₛᵢₒₙ_{_}_{region}=sin⁻¹ (n_{E}/n_{C}).

In various embodiments, treatment section 105 of medical treatment device 100 may emit the electromagnetic radiation inwards (e.g., from concave portion 102B), outwards (e.g., from convex portion 102A) and/or in other directions with respect to the direction of bending.

Optical fiber 110 may be a single-mode (e.g., designed for transmission of a single ray or mode of electromagnetic radiation) or a multi-mode (e.g., designed to carry multiple rays or modes of electromagnetic radiation simultaneously, each at a slightly different reflection angle within the optical fiber). In the latter case, power of electromagnetic radiation travelling along optical fiber 110 may be transferred to higher order modes that may leak out of the fiber. In various embodiments, emission region 145 and/or bending threshold are determined with respect to the required modes to induce lateral emission of the electromagnetic radiation in emission region 145 subsequently at the beginning of curved region 102.

In some embodiments, the refractive index n_{E} of element 140 is higher than the refractive index n_{C} of optical fiber 110 such that lateral emission may occur upon minimal bending of curved region 102, for example, through a substantially straight emission region 145 of treatment section 105.

The lateral emission may depend on various treatment section 105 and/or electromagnetic radiation characteristics that may comprise a size, a structure and/or materials of optical fiber 110, cover 120, element 140, bending radius 101, radiation frequency and/or radiation intensity, etc. Treatment section 105 may enable lateral emission in predetermined regions (e.g., emission regions 145) and/or may prevent the emission in other parts of the medical treatment device.

FIGS. 2A-2E are high level schematic illustrations of a system 200 for production of a medical treatment device 100 with a treatment section 105, according to some embodiments of the invention. FIG. 2A provides a side isometric view, FIG. 2B provides a side view, FIG. 2C provides a top isometric view and FIG. 2D provides a front view of system 200. It is noted that FIGS. 2A-2D illustrate system 200 without optical fiber 110. FIG. 2E provides an enlarged isometric view of optical fiber 110 and a glass member 290 positioned in system 200.

System 200 may enable bending treatment section 105 into a predetermined shape (e.g., curved portion 102 as shown in FIG. 1B). Optionally, system 200 may enable forming cover 120 over treatment section 105 of medical treatment device 100, by, for example, pressing a heated glass member (e.g., glass member 290 as described in detail with respect to FIG. 2E) to simultaneously attach glass member 290 to optical fiber 110 positioned within glass member 290 and/or bend treatment section 105. In some embodiments, glass member 290 is an elongated glass tube. In various embodiments, glass member 290 comprises element 140 affixed to its inner portion and/or element 140 is inserted between glass member 290 and optical fiber 110 prior heating (e.g., by heating element 230). In various embodiments, optical fiber 110 may be made from, for example, glass or quartz. Optionally, optical fiber 110 may be made from, for example, a polymer material. In some embodiments, optical fiber 110 and glass member 290 may comprise materials having, for example, similar mechanical properties, to, for example, enable their fusion.

System 200 may comprise an anchoring unit 210. Anchoring unit 210 may be configured to attach system 200 to, for example, a work station (not shown) using any connection means known in the art (e.g., bolts, screws, etc.).

System 200 may comprise first and/or second positioning elements 222, 224 (e.g., as shown in FIGS. 2A-2D). First and/or second positioning elements 222, 224 may be configured to receive and/or position optical fiber 110, and optionally a glass member 290, in a desired location in system 200 (e.g., as shown in FIG. 2E).

First and/or second positioning elements 222, 224 may be positioned at a predetermined distance 201 from each other along a longitudinal axis (e.g., X axis as shown in FIG. 2A) of system 200. Distance 201 may be predetermined based on, for example, a desired length of treatment section 105 of optical fiber 110. First and/or second positioning elements 222, 224 may comprise openings 222a, 224a, respectively. Openings 222a, 224a may have a shape and/or a size that may enable insertion and/or positioning of optical fiber 110, and optionally glass member 290, in a desired location in system 200 (e.g., as shown in FIG. 2E).

System 200 may comprise a heating element 230 (e.g., as show in FIG. 2D-2E). Heating element 230 may be configured to elevate a temperature of optical fiber 110 to a predetermined temperature threshold to enable bending. The temperature threshold may be predetermined to provide a desired softening to optical fiber 110. In various embodiments, heating element 230 is an oxygen-butane torch, a CO₂ laser source and/or conduction heating element. System 200 may comprise a heat shield 235 configured to prevent elevation of a temperature of optical fiber 110 outside treatment section 105.

Optionally, heating element 230 may enable fusion of optical fiber 110 with glass member 290 to form cover 120 over treatment section 105 (e.g., as shown in FIGS. 1A-1C). In various embodiments, multiple heating elements 230 are used to induce symmetric and/or asymmetric fusion of glass member 290 with optical fiber 110, while taking into account gravity effects.

System 200 may comprise at least one bending element configured to bend treatment section 105 of optical fiber 110 into a predetermined shape. In various embodiments, treatment section 105 is bent (e.g., by the at least one bending element) in one or more different spatial planes and/or along one or more axes. For example, treatment section 105 may be bent (e.g., by the at least one bending element) into a spiral shape.

System 200 may comprise, for example, a first bending element 242 and/or a second bending element 244, as shown in FIGS. 2A-2E. First and/or second bending elements 242, 244 may be configured to shape treatment section 105 of optical fiber 110 into a desired shape. First and/or second bending elements 242, 244 may be positioned between first and/or second positioning elements 222, 224 (e.g., as shown in FIGS. 2A-2E). First bending element 242 may comprise concave portions 242a and/or convex portions 242b and/or second bending element 244 may comprise convex portions 244a and/or concave portions 244b along the longitudinal axis of system 200. Concave portions 242a of first bending element 242 may correspond to convex portions 244a of second bending element 244 and/or convex portions 242b of first bending element 242 may correspond to concave portions 244b of second bending element 244. A shape and/or size of concave and convex portions 242a, 242b of first bending element 242 and corresponding convex and concave portions 244a, 244b of second bending element 244 may be predetermined based on, for example, the desired shape treatment section 105 of optical fiber 110 (e.g., curved portion 102 as shown in FIGS. 1B-1C).

First and/or second bending elements 242, 244 may comprise indents 242c, 244c. A shape and/or size of indents 242c, 244c may correspond to shape and/or size of optical fiber 110, and optionally to shape and size of glass member 290. First and/or second bending elements 242, 244 may be configured to move in a perpendicular direction with respect to the longitudinal axis of system 200 (e.g., along a Y axis and as indicated by dashed arrows in FIG. 2A) to press optical fiber 110 over treatment section 105, thereby bending treatment section 105 into the desired shape. Optionally, first and/or second bending elements 242, 244 may be configured to simultaneously press and/or cool heated glass member 290 to attach it to optical fiber 110, thereby forming cover 120 over treatment section 105 of the fiber, and/or may be further configured to bend treatment section 105 into the desired shape (e.g., curved portion 102 as shown in FIGS. 1B-1C). Optionally, the pressing may be configured to leave an air gap between optical fiber 110 and glass member 290 by, for example, controlling positive and/or negative pressure inside glass member 290. As may be apparent to one of ordinary skill in the art, while FIGS. 2A-2E illustrate two bending elements (e.g., first bending element 242 and second bending element 244), it is not meant to be limiting in anyway and system 200 may comprise any number of bending elements that may bend treatment section 105 into various shapes in various planes and/or along various axes (e.g., as described above).

In some embodiments, system 200 is configured only to fuse glass member 290 with optical fiber 110 over treatment section 105 (e.g., using heating elements 230). In some embodiments, system 200 is configured only to bend treatment section 105 (e.g., which comprises pre-fused glass member 290 and optical fiber 110) by the at least one bending element.

In various embodiments, system 200 is configured to fuse glass member 290 with optical fiber 110 over treatment section 105 at a first stage and bend treatment section 105 into a predetermined shape at a second stage and/or bend glass member 290 and optical fiber 110 into the predetermined shape at a first stage and fuse glass member 290 with optical fiber 110 at a second stage. In some embodiments, system 200 is configured to simultaneously fuse glass member 290 with optical fiber 110 over treatment section 105 and bend treatment section 105 into a predetermined shape.

Optionally, a portion of a distal end of treatment section 105 (e.g., a portion positioned within second positioning element 224 which was not bent) may be cut using, for example, a scriber (not shown). In some embodiments, the cut end of treatment section 105 may be heated (e.g., by heating element 230) to form cap 122 (e.g., as shown in FIG. 1A.

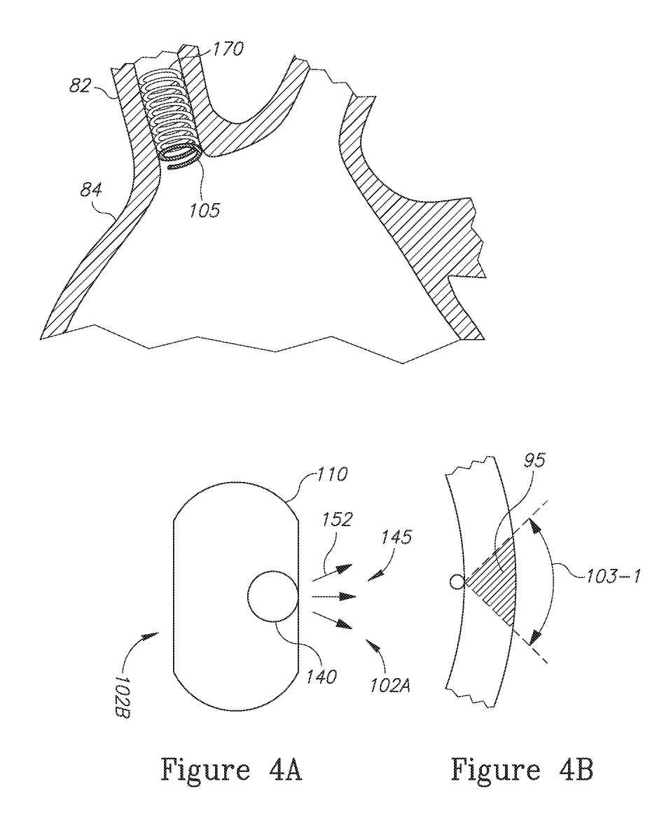

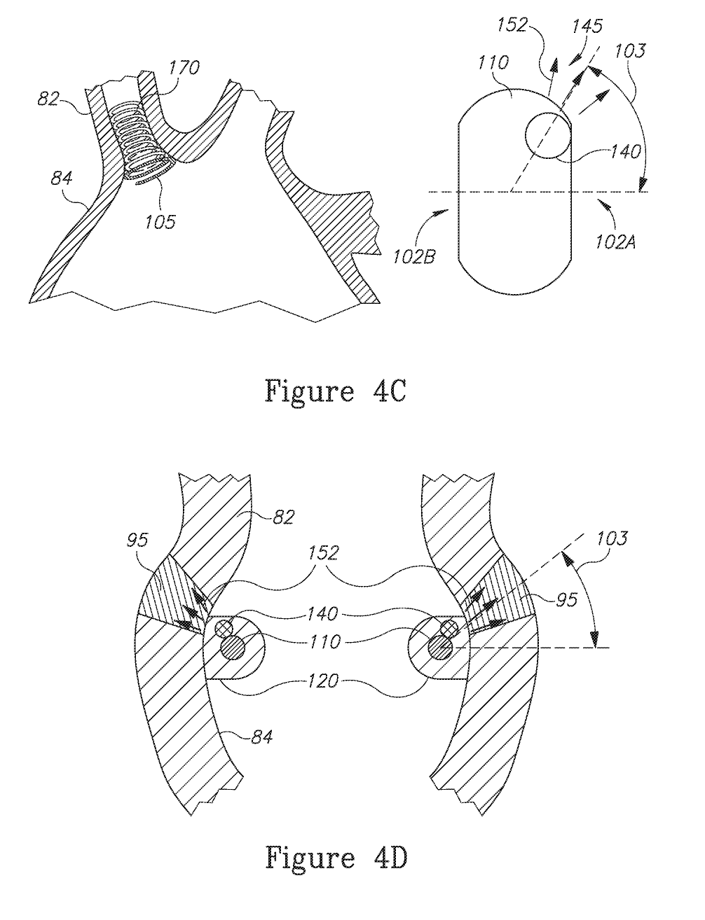

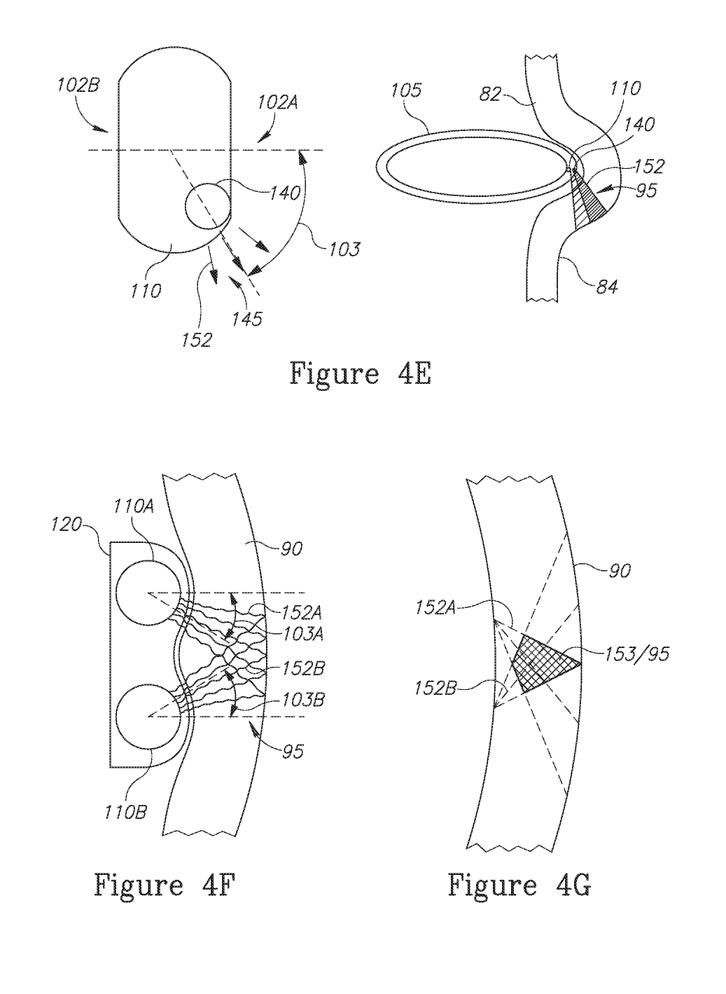

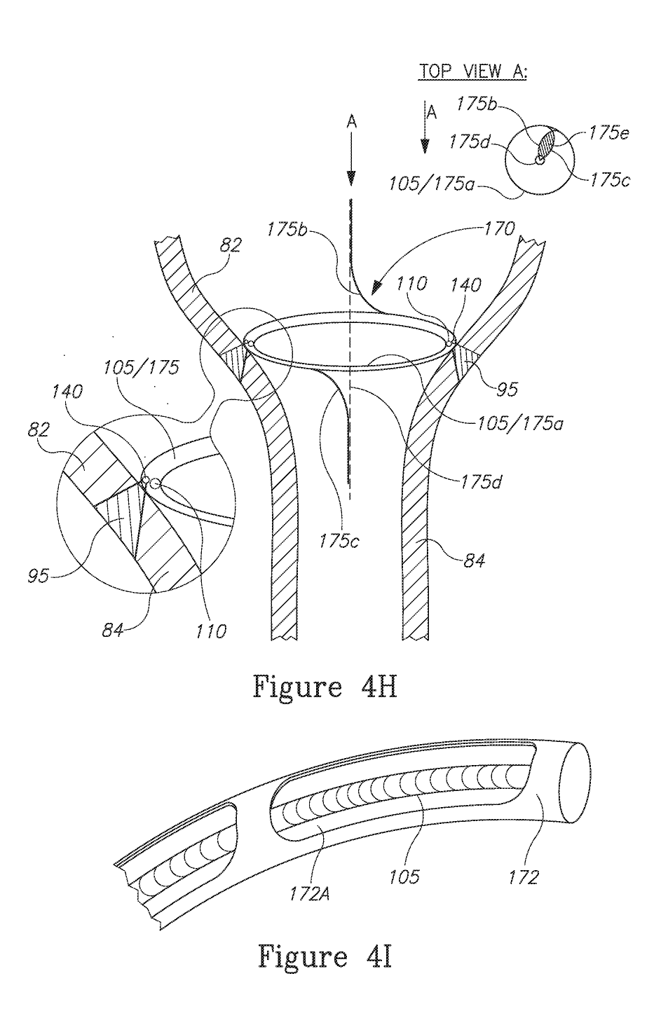

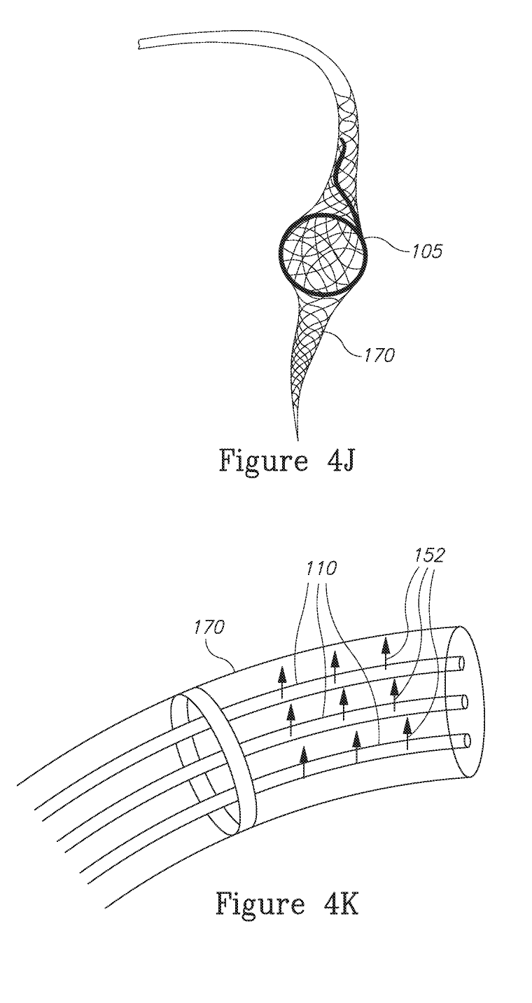

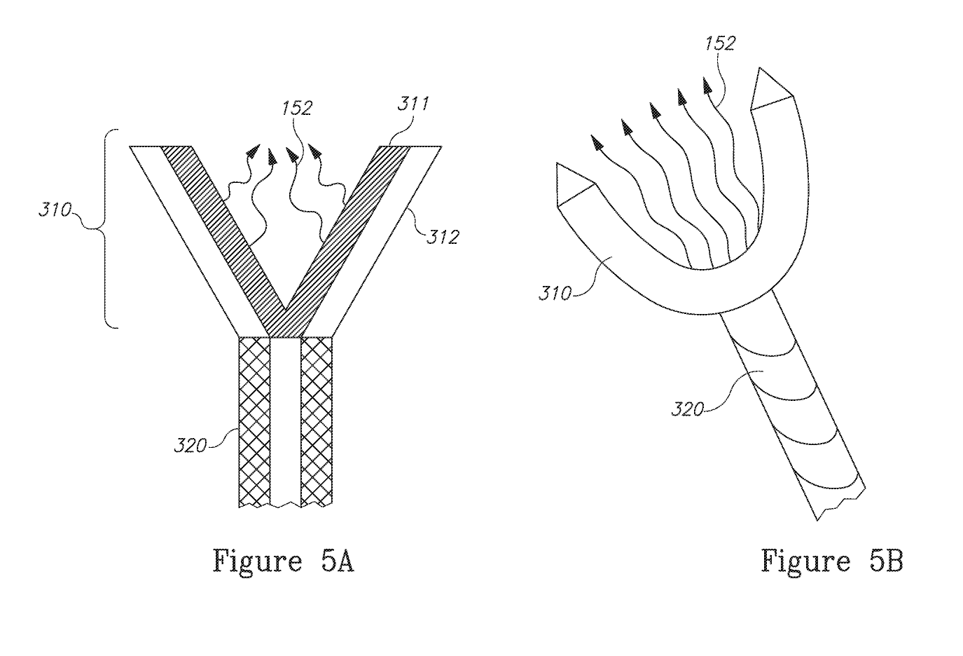

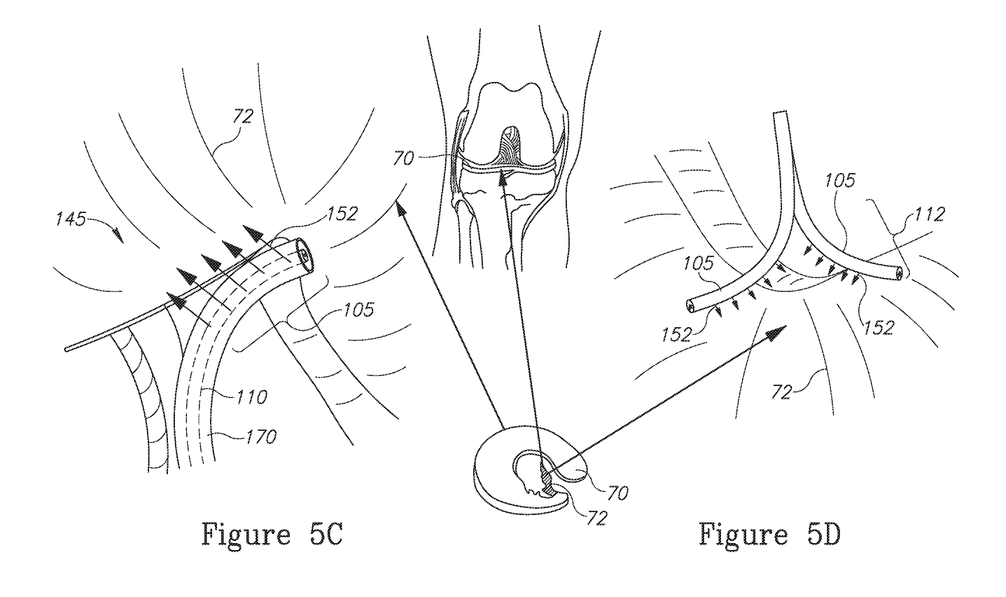

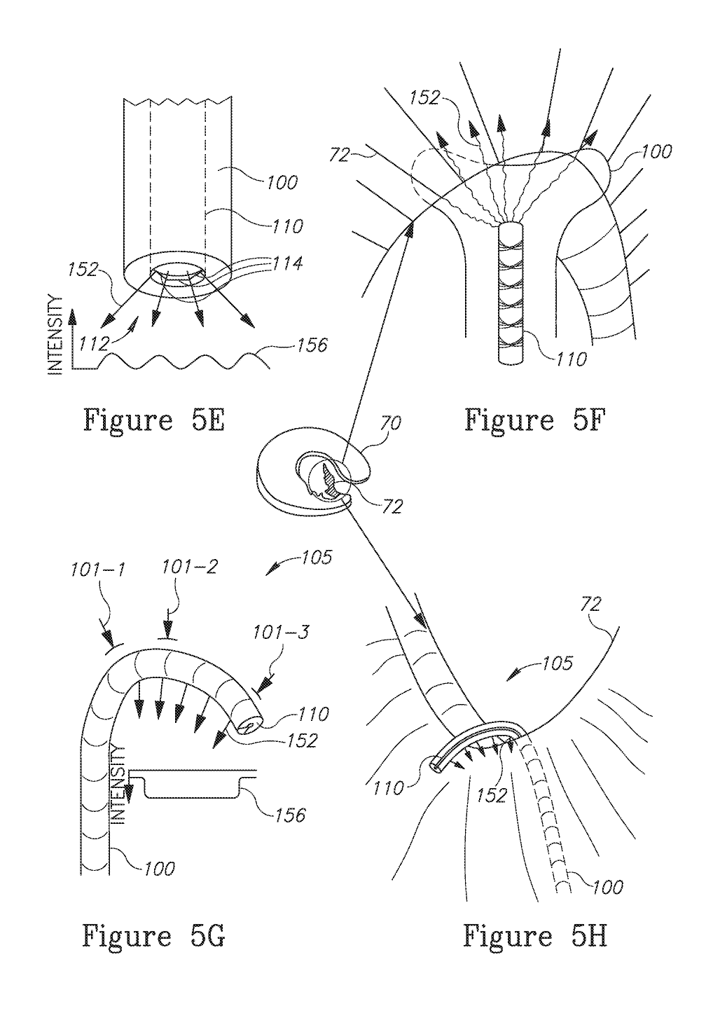

In embodiments, one and/or more optical fibers 110 having one or more treatment section 105 may be incorporated in medical treatment device 100 that may comprise at least one electromagnetic source to transmit electromagnetic radiation through one and/or more treatment sections 105. The following description starts with embodiments of treatment section 105 and continues with embodiments of medical treatment device 100. In various embodiments, any one of treatment section 105 embodiments (e.g., as shown in FIGS. 1-3) may be implemented in any one of medical treatment devices 100 embodiments (e.g., as shown in FIGS. 4, 5) and all possible combinations are comprised therefore in the present invention.

FIGS. 3A-3M are high level schematic illustrations of various embodiments of treatment section 105 of medical treatment device 100, according to some embodiments of the invention. FIGS. 3A-3J and FIGS. 3L-3M illustrate cross-sectional views of treatment section 105.

FIGS. 3A-3C and FIG. 3M illustrate treatment section 105 where optical fiber 110 is fused with cover 120 along a whole circumference of the fiber. Optical fiber 110 may have a substantially D-shape cross-section that may comprise at least one flat portion 116A and/or at least one curved portion 116B. Optical fiber 110 may be positioned concentrically within cover 120. An inner portion of cover 120 may have matching D-shape cross-section to enable fusing of optical fiber 110 with cover 120 along the whole circumference of the fiber. In various embodiments, D-shape cross-section of optical fiber 110 and/or of cover 120 is generated during heating and/or fusion of glass member 290 and optical fiber 110 (e.g., as described above with respect to FIGS. 2A-2E) due to, for example, surface tension forces and/or vacuum applied between the optical fiber and the glass member. Flat regions 116A may also be indicators of a desired positioning and/or orientation of optical fiber 110 within cover 120 during the production process.

In some embodiments, treatment section 105 comprises elements 140 positioned within optical fiber 110 at predetermined locations. Treatment section 105 may comprise two elements 140 (e.g., as shown in FIGS. 3A-3B), three elements 140 (e.g., as shown in FIG. 3C) and/or any number of elements 140. Elements 140 may be positioned adjacent (e.g., as shown in FIGS. 3B-3C) and/or at a predetermined distance 142 from each other (e.g., as shown in FIG. 3A). Elements 140 may be positioned at an interface between optical fiber 110 and cover 120 (e.g., as shown in FIGS. 3A-3D). In various embodiments, elements 140 are positioned adjacent to flat region 116A of optical fiber 110 at convex portion 102A (e.g., as shown in FIG. 3A) and/or adjacent to curved portion 116B (e.g., as shown in FIG. 3C). In some embodiments, elements 140 are positioned adjacent to an intersection of flat and curved portions 116A, 116B of optical fiber 110 at convex portion 102A of treatment section 105 (e.g., as shown in FIG. 3B). In various embodiments, a cross-sectional shape of elements 140 may be circular, oval, rectangular, triangular or may have any other shape.

Additionally or alternatively, element 140 may comprise a covering 141 that may envelope element 140 (e.g., as shown in FIG. 3M). In various embodiments, covering 141 may be tangent to outer surface of optical fiber 110 and/or tangent to interface between optical fiber 110 and cover 120.

FIGS. 3D-3E illustrate treatment section 105 where optical fiber 110 is fused with cover 120 along a portion of circumference of the fiber. In various embodiments, optical fiber 110 is positioned eccentrically within cover 120 and/or fused with cover 120 at concave portion 102B of the treatment section 105. In some embodiments, optical fiber 110 is fused with cover 120 at convex portion 102A (not shown). Treatment section 105 may comprise an air gap 125 between non-fused portions of optical fiber 110 and cover 120.

FIG. 3D illustrates optical fiber 110 having a D-shape cross-section including flat portion 116A and/or curved portion 116B. Flat portion 116A may, for example, facilitate positioning of optical fiber 110 within cover 120 upon bending of desired portion 102 of treatment section 105, as described above. In some embodiments, element 140 is positioned adjacent to curved portion 116B of optical fiber 110 at concave portion 102B of treatment section 105.

FIG. 3E illustrates optical fiber 110 having substantially circular cross-section. Optical fiber 110 may be fused with cover 120 at concave region 102B of treatment section 105.

FIG. 3F illustrates treatment section 105 including two optical fibers 110A, 110B, air gap 125 and element 140 embedded within cover 120. Optical fibers 110A, 110B may be fused with cover 120 along the whole circumference of the fibers (e.g., as shown in FIG. 3F) and/or along a portion of the circumference (e.g., as shown in FIG. 3E). Optical fibers 110A, 110B may have circular cross-section (e.g., as shown in FIG. 3F) and/or D-shape cross-section (e.g., as shown in FIGS. 3A-3D). Element 140 may have a substantially circular shape in the cross-section and/or may at least partly enclose optical fibers 110A, 110B and/or air gap 125. In some embodiments, optical fibers 110A, 110B are positioned adjacent to element 140 (e.g., as shown in FIG. 3F). Optical fibers 110A, 110B may be positioned at opposite portions of cover 120 (not shown) and/or may be positioned at the same portion at a predetermined angle to each other (e.g., as shown in FIG. 3F).

FIG. 3G illustrates treatment section 105 with element 140 that varies its position along a longitudinal axis 104 of a medical treatment device 100. Element 140 may be positioned within optical fiber 110 in treatment section 105 along a substantially helical path around longitudinal axis 104. For example, at the beginning of curved region 102 of treatment section 105, element 140 may be positioned adjacent to curved portion 116B of optical fiber 110 (e.g., cross-section A-A as shown in FIG. 3G). At the mid of curved region 102, element 140 may be positioned adjacent to an intersection of flat and/or curved portions 116A, 116B of optical fiber 110 at convex portion 102A (e.g., cross-section B-B as shown in FIG. 3G). At the end of curved region 102, element 140 may be positioned adjacent to flat portion 116A of optical fiber 110 at concave portion 102B (e.g., cross-section C-C as shown in FIG. 3G).

A spatial configuration of optical fiber 110 within treatment section 105 (e.g., bending of treatment section 105 beyond a predetermined bending threshold to provide curved region 102), a spatial configuration of elements 140 within treatment section 105 (e.g., number of elements 140, position of elements 140 in a cross-section of treatment section 105 with respect to direction of bending, distance 142 between adjacent elements 140, etc.) and/or optical properties of optical fiber 110, element 140 and optionally of cover 120 (e.g., refractive indexes n_{C}, n_{E}, n_{P} and/or n_{F}) may determine emission region 145 in treatment section 105. In some embodiments, emission region 145 is defined (e.g., based on a spatial configuration of optical fiber 110 within treatment section 105, a spatial configuration of elements 140 within treatment section 105 of medical treatment device 100 and/or optical properties of optical fiber 110, element 140 and/or cover 120) to enable emission of a predetermined amount of electromagnetic radiation (e.g., at least 70%) travelling along optical fiber 110 through, for example, concave portion 102B of curved region 102 of treatment section 105.

In some embodiments, emission region 145 is determined to be associated with elements 140 (e.g., as described above) to emit electromagnetic radiation 152 through elements 140. For example, treatment section 105 may be configured to emit electromagnetic radiation 152 radially outwards from convex portion 102A (e.g., in a plane determined by treatment section 105) through emission region 145, as shown in FIG. 3H. Emission region 145 may be associated with, for example, elements 140, that may be positioned, for example, adjacent to flat portion 116A at convex portion 102A of treatment section 105 (e.g., as shown in FIG. 3A and FIG. 3H).

Treatment section 105 may be configured to emit electromagnetic radiation 152 outwards and at a predetermined angle 103 with respect to the direction of bending (e.g., with respect to the plane determined by treatment section 105) through emission region 145, as shown, for example, in FIG. 3I. Emission region 145 may be associated with, for example, elements 140 that may be positioned, for example, at an intersection of flat and curved portion 116A, 116B at convex portion 102A (e.g., as shown in FIG. 3B and FIG. 3I).

In some embodiments, treatment section 105 is configured to emit electromagnetic radiation 152 in a direction being perpendicular to the direction of bending (e.g., at predetermined angle 103 of 90 degrees) through emission region 145, as shown in FIG. 3J. Emission region 145 may be associated with, for example, elements 140 that may be positioned, for example, adjacent to curved portion 116B of optical fiber 110 (e.g., as shown in FIGS. 3C, 3J).

Reference is now made back to FIGS. 3D-3G. In some embodiments, treatment section 105 is configured to emit electromagnetic radiation 152 inwards with respect to direction of bending from concave portion 102B through emission region 145, for example as shown in FIGS. 3D-3E. In various embodiments, emission region 145 may be associated with, for example, element 140 that may be positioned, for example, at concave portion 102B of optical fiber (e.g., as shown in FIG. 3D) and/or emission region 145 may be determined by, for example, bending of treatment section 105 beyond a predetermined bending threshold (e.g., in the production process) and/or by tailoring optical properties of optical fiber 110 (e.g., as shown in FIG. 3E).

Treatment section 105 may be configured to comprise two emissions regions, 145A, 145B, for example, as shown in FIG. 3F. Emission regions 145A, 145B may be associated with optical fibers 110A, 110B, respectively and/or may be determined as described above. Emission regions 145A, 145B may have similar or different emission characteristics. For example, emission regions 145A, 145B may emit electromagnetic radiation 152A, 152B with different parameters (e.g., wavelength and/or intensity). In some embodiments, electromagnetic radiation is delivered to fibers 110A, 110B simultaneously or sequentially.

Treatment section 105 may be configured to emit electromagnetic radiation 152 through emission region 145 that varies its location along longitudinal axis 104 of medical treatment device 100, for example, as shown in FIG. 3G. Emission region 145 may be associated with elements 140 (e.g., as described above) that may be positioned in optical fiber 110 along a substantially helical path around longitudinal axis 104 as described in detail with respect to FIG. 3G. For example, at the beginning of curved portion 102 treatment section 105 may emit electromagnetic radiation 152 through emission region 145 in a perpendicular direction with respect to bending direction (e.g., cross-section A-A, as shown in FIG. 3G). At the mid of curved region 102, treatment section 105 may emit electromagnetic radiation 152 through emission region 145 at predetermined angle 103 with respect to direction of bending (e.g., cross-section B-B, as shown in FIG. 3G). At the end of curved region 102, treatment section 105 may emit electromagnetic radiation 152 inwards with respect to direction of bending through emission region 145 (e.g., cross-section C-C, as shown in FIG. 3G).

It is noted that treatment sections 105 illustrated in FIGS. 3A-3J may also be designed to emit lateral radiation in a non-bended configuration of the medical treatment device (e.g., through a substantially straight emission region 145 in treatment section 105), by, for example, tailoring the refractive index n_{E} of elements 140 to be higher than the refractive index n_{C} of optical fiber 110.

FIGS. 3K-3L illustrate treatment section 105 having optical fiber 130 with a core 136 and an asymmetric cladding 137, according to some embodiments of the invention. FIG. 3K and FIG. 3L provide isometric and cross-sectional views of treatment section 105, respectively.

Optical fiber 130 may comprise an asymmetric cladding. The asymmetric cladding may comprise differing cladding types 137A, 137B, 137C, 137D having, for example, differing refractive indexes. Optionally, cover 120 may be affixed to optical fiber 130 over treatment section 105 of medical treatment device 100. Treatment section 105 may comprise emission regions 145A, 145B, 145C associated with claddings 137A, 137B, 137C, respectively, which are different from cladding 137D in non-emitting regions (e.g., as shown in FIG. 3I). Treatment section 105 may comprise cover 120 (e.g., that may be fused with optical fiber 130, as described above with respect to FIGS. 2A-2E).

Optical fiber 130 having the asymmetric cladding (e.g., cladding types 137A, 137B, 137C) may be configured to laterally emit electromagnetic radiation (e.g., as show in FIGS. 3K-3L) upon bending of the fiber beyond a predetermined bending threshold, as described in detail in U.S. Patent Application No.: US2014/0288541A1, which is incorporated herein by reference.

In some embodiments, emission regions 145A, 145B, 145C may be different from each other in the characteristics of emitted electromagnetic radiation 152A, 152B, 152C, respectively. For example, the characteristics of electromagnetic radiation may comprise different wavelength ranges and/or different intensities.

FIGS. 1-3 illustrate one, two and/or three emission regions 145 as examples. In various embodiments, treatment section 105 comprises a plurality of emission regions 145 at different predetermined locations along the assembly and/or any region and/or location configuration according to given requirements.

In various embodiments, optical fiber 110 and/or inner portion of cover 120 have substantially circular cross-section (e.g., as shown in FIGS. 3E-3F), substantially D-shape cross-section (e.g., as shown in FIGS. 3A-3D) and/or any other shape. The shape of the cross-section may, for example, facilitate positioning of optical fiber 110 within the cover 120 upon bending a desired portion of treatment section 105 and/or to determine lateral emission beam shape.

In various embodiments, optical fiber 110 has a uniform index profile (e.g., as shown in FIGS. 3A-3J), a circumferentially symmetric index profile that may vary along a radius of the fiber and/or asymmetric index profile that may vary along a radius and/or circumference of the fiber (e.g., as shown in FIGS. 3K-3L).

In embodiments, medical treatment device 100 comprises a supportive structure 170, as shown, for example in FIGS. 4A-4K. Supportive structure 170 may comprise at least one of: a guide-wire, a balloon, a stent, a nitinol tube, a catheter tube, a loop, a spiral or any combination thereof In various embodiments, one or more optical fibers 110 may be coupled to and/or incorporated within supportive structure 170. In some embodiments, a portion of optical fiber 110, for example treatment section 105, is coupled to supportive structure 170.

Supportive structure 170 may be used to position and/or to orient treatment section 105 of medical treatment device 100 with respect to a target region 95 such that emission region 145 may laterally emit electromagnetic radiation upon the target. In some embodiments, supportive structure 170 enables good contact of treatment section 105 with target region 95 and/or with tissue surrounding target region 95. Supportive structure 170 may be associated in various embodiments of medical treatment device 100, as described in detail below with respect to FIG. 4 and FIG. 5.

FIGS. 4A-4B are high level schematic illustrations of a medical treatment device 100 having a treatment section 105 coupled to a supportive structure 170 and designed to emit electromagnetic radiation from convex portion 102A of treatment section 105 upon a target region 95, according to some embodiments of the invention. FIG. 4A provides a front view of medical treatment device 100 (at the top of FIG. 4A) and cross-sectional view of treatment section 105 (at the bottom of FIG. 4A). FIG. 4B provides cross-sectional view of medical treatment device 100 and target region 95.

In various embodiments, medical treatment device 100 is used to treat various fibrillation and/or arrhythmia diseases and/or to generate conduction blocks in at least one of: a pulmonary vein, a left atrium, an intersection region of the pulmonary vein and the left atrium, a left ventricle and a right ventricle. In some embodiments, medical treatment device 100 is used, for example, to treat an atrial fibrillation disease. The treatment of the atrial fibrillation disease may comprise, for example, scaring and/or ablation of a tissue at an intersection of a pulmonary vein 82 with a left atrium 84 of a heart 80 (e.g., target region 95, as shown in FIGS. 4A-4H). Prior art treatment of the atrial fibrillation disease typically comprises a radiofrequency (RF) ablation of target region 95. One disadvantage of the RF ablation may comprise lack and/or limited control and/or feedback features that may lead, for example, to an insufficient ablation of target region 95 and as a result to a recurrence of the atrial fibrillation. The RF ablation may also lead to over ablation that may, for example, damage tissue and/or organs surrounding target region 95 and as a result may cause, for example, bleeding, perforations and/or occurrence of thromboses. Another disadvantage of the RF ablation is that it cannot be performed along the whole circumference of target region 95 in a single treatment step.

In some embodiments, supportive structure 170 of medical treatment device 100 is a guide wire (e.g., as shown in FIGS. 4A, 4C, 4H). Optical fiber 110 and/or treatment section 105 may be embedded and/or coupled to a tip of guide wire 170. Guide wire 170 may be inserted, for example, to pulmonary vein 82 and/or may be used to position treatment section 105 of medical treatment device 100 at correct position and/or orientation with respect to, for example, the intersection of pulmonary vein 82 and left atrium 84 (e.g., target region 95) and/or to provide optimal contact between treatment section 105 and a tissue surrounding target region 95. Treatment section 105 may be designed as a bended ring-like structure, for example as shown in FIGS. 4A, 4C, 4H.

Treatment section 105 may be designed to laterally emit electromagnetic radiation 152 from convex portion 102A of the section through emission region 145. In various embodiments, treatment section 105 comprises optical fiber 110 having, for example, D-shape cross-section and/or element 140 positioned at convex portion 102A (e.g., adjacent to flat portion 116A) of the treatment section (e.g., as shown in FIG. 4A and as described in detail with respect to FIGS. 3A, 3H). Optionally, treatment section 105 comprises cover 120 attached to optical fiber 110. In various embodiments, cover 120 is made of deformable and/or flexible material. Deformable and/or flexible cover 120 may allow adjusting treatment section 105 to provide correct positioning, orientation and/or optimal contact of the treatment section with a tissue surrounding target region 95. In some embodiments, treatment section 105 is provided without cover 120.

In some embodiments, emission region 145 is defined (e.g., based on a spatial configuration of optical fiber 110 within treatment section 105, a spatial configuration of elements 140 within treatment section 105 of medical treatment device 100 and/or optical properties of optical fiber 110, element 140 and optionally of cover 120) to enable emission of a predetermined amount of electromagnetic radiation (e.g., at least 70%) travelling along optical fiber 110 through, for example, convex portion 102A of curved region 102 of treatment section 105.

Treatment section 105 may be positioned, for example, concentrically within pulmonary vein 82 (e.g., using guide wire 170) at a level of the intersection of the vein with atrium 84 (e.g., at a level of target region 95) such that convex portion 102A of the treatment section overlaps with target region 95 along the whole circumference of the target, for example as shown in FIG. 4A. Accordingly, treatment section 105 may emit electromagnetic radiation 152 from convex portion 102A upon target region 95 through, for example, emission region 145. The ring-like structure of treatment section 105 which, for example, overlaps with target region 95 may allow scarring and/or ablation of target region 95 along the whole circumference of the target in a single treatment step. A depth of the scarring and/or ablation may be controlled, for example, by emitting electromagnetic radiation at a predetermined wavelength. For example, emitting electromagnetic radiation at a wavelength in a range of 100-2100 nm may result in ablation of target region 95 through a whole thickness of the tissue due to, for example, minimal absorption of the emitted radiation by water contained within the tissue.

In various embodiments, a desired shape of a lateral emission beam (for example, angle 103-1 as shown in FIG. 4B) is predetermined by location and/or number of elements 140 within optical fiber 110 or optionally within cover 120.

FIGS. 4C-4E are high level schematic illustrations of a medical treatment device 100 having a treatment section 105 coupled to a supportive structure 170 and designed to emit electromagnetic radiation at a predetermined angle 103 with respect to direction of bending upon a target region 95, according to some embodiments of the invention. FIG. 4C provides a front view of medical treatment device 100 (at the top of FIG. 4C) and cross-sectional view of treatment section 105 (at the bottom of FIG. 4C). FIGS. 4D-4E provide cross-sectional view of medical treatment device 100 and target region 95.

Treatment section 105 may be designed to laterally emit electromagnetic radiation 152 through emission region 145 at predetermined angle 103 with respect to direction of bending (e.g., angled ablation). Treatment section 105 may comprise optical fiber 110 having, for example, D-shape cross-section and/or element 140 positioned at an intersection of flat and curved portion 116A, 116B at convex portion 102A of the treatment section such that electromagnetic radiation 152 may be emitted at predetermined angle 103 with respect to direction of bending, as shown in FIGS. 4C-4D and as described in detail with respect to FIG. 3B, 3I. Treatment section 105 may be positioned (e.g., using guide wire 170), for example, within left atrium 84 and emit electromagnetic radiation 152 upon target region 95 (e.g., the intersection of pulmonary vein 82 and left atrium 84) from an interior portion of the atrium, as shown in FIG. 4D. Guide wire 170 may be used to position and/or orient treatment section 105 and/or emission region 145 to emit in a desired direction upon target region 95.

In various embodiments, predetermined angle 103 of lateral emission of electromagnetic radiation 152 is controlled by predetermined location of elements 140 within optical fiber 110 or optionally within cover 120. For example, as shown in FIG. 4E, positioning of element 140 at an opposite intersection of flat and curved portion 116A, 116B (e.g., as compared to FIG. 4C) at convex portion 102A of treatment section 105 may result in lateral emission in opposite direction, as compared to shown in FIG. 4C.

FIG. 4F is a high level schematic illustration of a medical treatment device 100 with a treatment section 105 having two emission regions 145A, 145B, according to some embodiments of the invention. FIGS. 4F provides a cross-sectional view of treatment section 105 and target region 95. FIG. 4G illustrates an intersection zone 153 generated by electromagnetic radiations 152A, 152B emitted from emission regions 145A, 145B of medical treatment device 100, according to some embodiments of the invention. FIG. 4G presents a cross-sectional view of an intersection zone 153.

Medical treatment device 100 and/or treatment section 105 may comprise, for example, two optical fibers 110A, 110B. Optionally, optical fibers 110A, 110B may be embedded in cover 120, as shown, for example, in FIG. 4F. In various embodiments, cover 120 is made of deformable and/or flexible material. Deformable and/or flexible cover 120 may allow delivery of treatment section 105 through a body of a patient (e.g., in a compact package) to target region 95, deployment of treatment section 105 with respect to target region 95 and/or further adjustment of treatment section 105 to provide correct positioning, orientation and/or optimal contact of the treatment section with a tissue surrounding target region 95. In some embodiments, cover 120 enables coupling of optical fibers 110A, 110B to medical treatment device 100.Treatment section 105 may comprise emission regions 145A, 145B associated with optical fibers 110A, 110B, respectively, as shown in FIG. 4F. The emission regions 145A, 145B may be determined as described in detail with respect to FIGS. 3A-3L.

In some embodiments, emission regions 145A, 145B configured to emit electromagnetic radiation 152A, 152B upon target region 95 within a tissue 90 (for example, the intersection of pulmonary vein 82 and left atrium 84 of heart 80, as described in detail with respect to FIGS. 4A-4E) at predetermined angles 103A, 103B with respect to direction of target (e.g., as shown in FIG. 4F). In some embodiments, angles 103A, 103B are predetermined such that electromagnetic radiations 152A, 152B emitted from emission regions 145A, 145B, respectively, generate an intersection zone 153 within target region 95 where most of the emitted electromagnetic energy is concentrated (e.g., as shown in FIGS. 4G). Accordingly, scarring and/or ablation of tissue 90 may occur within intersection zone 153 of target region 95, thus preventing, for example, a damage to a surface of tissue 90 surrounding the target thereby preventing, for example, occurrence of thromboses.

FIGS. 4H-4K are high level schematic illustrations of various embodiments of supportive structures 170 of medical treatment device 100, according to some embodiments of the invention.

In some embodiments, medical treatment device 100 comprises treatment section 105 coupled to and/or embedded within at least a portion of supportive structure 170. Supportive structure 170 may be inserted into a body of a patient via, for example, a vessel (e.g., femoral and/or radial arteries) to deliver treatment section 105 to a desired target region 95 (e.g., intersection of pulmonary vein 82 and left atrium 84). In some embodiments, supportive structure 170 allows a continued blood flow through a lumen of the vessel. Supportive structure 170 may also be used to position and/or to orient treatment section 105 and/or emission region 145 of medical treatment device 100 with respect to a target 95 such that emission region 145 may laterally emit electromagnetic radiation 152 upon the target. In some embodiments, supportive structure 170 enables good contact of treatment section 105 with target 95 and/or surrounding tissue.

FIG. 4H presents supportive structure 170 having a looped portion 175a. Looped portion 175a may be generated by rotating a first end 175b of supportive structure 170 against a second end 175c of supportive structure 170 around a central axis 175d, where central axis 175d being substantially perpendicular to a plane of treatment region 95. In some embodiments, treatment section 105 may be affixed to looped portion 175a. In various embodiments, supportive structure 170 may comprise a region 175e in which first end 175b overlaps with second end 175c such that looped portion 175a and/or treatment section 105 affixed to looped portion 175a completes a loop of at least 360° around central axis 175d (e.g., as shown in View A in FIG. 4H).

In some embodiments, at least a tip 171 of supportive structure 170 (e.g., as shown in FIG. 4A and FIG. 4C) comprises a nitinol tube 172 having a predetermined shape, for example as shown in FIG. 4I. In some embodiments, treatment section 105 of medical treatment device 100 is embedded within nitinol tube 172. Nitinol tube 172 may comprise an opening 172A to enable lateral emission of electromagnetic energy 152 from emission region 145 of treatment section 105. A position of opening 172A in nitinol tube 172 may be predetermined to allow lateral emission of electromagnetic energy in a desired direction upon target region 95.

FIG. 4J presents supportive structure 170 as a stent-like structure. In some embodiments, one or more optical fibers 110 and/or treatment section 105 are incorporated in stent 170. For example, optical fibers 110 and/or treatment section 105 may be wound around stent 170 and/or may replace one or more of the wires of stent 170. Treatment section 105 may be bent into a ring-like shape and/or coupled to stent 170, as shown in FIG. 4J. Stent 170 may be inserted, for example, into pulmonary vein 82 and may position and/or orient treatment section 105 and/or emission region 145 to laterally emit electromagnetic radiation upon at target region 95 (e.g., the intersection of pulmonary vein 82 and left atrium 84) along a whole circumference of the target and/or in a single treatment step.

FIG. 4K presents supportive structure 170 as a compliant balloon. Optical fibers 110 and/or treatment sections 105 may be positioned on external surface of balloon 170. For example, optical fibers 110 and/or treatment section 105 may be positioned in a transverse or a longitudinal direction with respect to a length axis of balloon 170 and/or exhibit any combination thereof or other form. In some embodiments, optical fibers 110 and/or treatment section 105 may be wound in a configuration that is perpendicular, parallel or oblique to a longitudinal axis of balloon 170 or in a combination thereof. FIG. 4K presents three treatment sections 105 coupled to external surface of balloon 170 in a longitudinal direction along the balloon.

One advantage of the present invention may comprise controlling a depth of a target region (e.g., the intersection of pulmonary vein 82 with left atrium 84) by controlling a dosage of emitted electromagnetic radiation (e.g., electromagnetic radiation 152) based on, for example, a closed loop temperature measurement (e.g., using a thermocouple, a measurement of a power of emitted electromagnetic radiation by, for example, a looping back fiber and/or calculating an index of refraction of tissue and/or of a water and thereby determining a temperature), using the optical fiber (e.g., optical fiber 110) of the medical treatment device and/or using various methods known in the art. Controlling the depth of the target region may prevent, for example, an insufficient ablation and/or over ablation of the tissue thereby optimizing the treatment. Another advantage of the present invention may comprise performing electrophysiological measurements while applying a treatment (e.g., emitting electromagnetic radiation 152) onto a target region (e.g., the intersection of pulmonary vein 82 with left atrium 84).

Another advantage of the present invention may comprise applying a treatment (e.g., emitting electromagnetic radiation 152) over a whole target region in a single treatment step. For example, ring-like treatment section 105 of medical treatment device may emit electromagnetic radiation 152 onto a whole circumference of a substantially circular target region (e.g., the intersection of pulmonary vein 82 with left atrium 84) in a single treatment step eliminating a need in shifting and/or reposition of the medical treatment device.

FIGS. 5A-5B are high level schematic illustrations of a medical treatment device 100 having a V-shape unit 310 and optical fiber delivery unit 320, according to some embodiments of the invention.

Medical treatment device 100 may comprise a V-shape unit 310 configured to emit electromagnetic radiation 152 upon target region 95. Medical treatment device 100 may also comprise an optical fiber delivery unit 320. In some embodiments, V-shape unit 310 is coupled to optical fiber delivery unit 320, for example as shown in FIGS. 5A-5B.

V-shape unit 310 may comprise one or more layers. For example, V-shape unit 310 may comprise a first layer 311 and/or a second layer 312, as shown in FIG. 5A. First and/or second layers 311, 312 may have a substantially V-shape cross-section. In various embodiments, first and/or second layers 311, 312 comprise different materials and/or have different refractive indexes. For example, first and/or second layers 311, 312 may have refractive indexes n_{V1}, n_{V2}, respectively. In some embodiments, the refractive index n_{V2} of second layer 312 is smaller than the refractive index n_{V1} of first layer 311 in order to, for example, focus and/or concentrate an emitted electromagnetic energy 152 within an inner portion of V-shape unit 310 (e.g., as shown in FIG. 5A).

In various embodiments, a transverse cross-section (e.g., cross-section that is perpendicular to a longitudinal cross-section shown in FIG. 5A) of V-shape unit 310 has a triangular shape that is designed to, for example, focus a mechanical energy and/or optical (e.g., electromagnetic) energy at a point of contact of V-shape unit 310 with a target region 95. Focusing of the mechanical and/or optical energy at the point of contact between V-shape unit 310 and target region 95 may, for example, increase the energy density in the point of contact which may, for example, increase the treatment efficiency. Medical treatment device 100 may be configured to emit electromagnetic energy 152, for example upon a contact of V-shape unit 310 and target region 95.

Optical fiber delivery unit 320 may comprise at least one optical fiber configured to deliver electromagnetic radiation to V-shape unit 310. The at least one optical fiber within optical fiber delivery unit 320 may be configured to emit electromagnetic radiation from, for example, a tip of the fiber, which is further delivered to V-shape unit 310 and emitted onto target region 95 as described above.

FIGS. 5C-5D are high level schematic illustrations of a medical treatment device 100 having at least one treatment section 105 at a distal end 112 of the device, according to some embodiments of the invention.

Medical treatment device 100 may comprise one treatment section 105 (e.g., as shown in FIG. 5C) and/or two treatment sections 105 arranged in a V-shape structure (e.g., as shown in FIG. 5D) at distal end 112 of the device, where each of treatment section 105 comprises at least one optical fiber 110. The bending of treatment sections 105 may be stationary (e.g., designed during the production process) or dynamic (e.g., established during the treatment procedure). In some embodiments, treatment sections 105 of medical treatment device 100 (e.g., as shown in FIGS. 5C-5D) comprise any of treatment section 105 embodiments shown in FIGS. 3A-3J. In some embodiment, treatment sections 105 of medical treatment device 100 (e.g., as shown in FIGS. 5C-5D) comprise any of treatment section 105 embodiments shown in FIGS. 3K-3L.

In various embodiments, medical treatment device 100 may be used, for example, in a meniscectomy, partial meniscectomy, various cartilage and/or ligaments removal and/or cutting procedures and/or prostatectomy. FIGS. 5C-5D, 5F, 5I illustrate medical treatment device 100 as the meniscectomy tool, according to some embodiments of the invention. Prior art meniscectomy tools typically comprise mechanical biters, shavers and/or electrical bi-polar scaring tools.

Medical treatment device 100 (e.g., devices shown in FIGS. 5A-5D) may be used, for example, to cut a torn portion 72 of a meniscus 70 (e.g., target region 95, as shown in FIGS. 5C-5D) and/or to weld torn portion 72 to meniscus 70. Welding of torn portion 72 to meniscus 70 may comprise applying a mechanical pressure (e.g., by treatment section 105) on portion 72 to establish a contact of the portion with the meniscus and/or emitting electromagnetic radiation 152 through emission region 145 at predetermined wavelength to, for example, elevate a temperature of torn portion 72 and/or meniscus 70 to a predetermined temperature level (e.g., 61-63° Celsius) to enhance welding. In some embodiments, a depth of penetration of electromagnetic radiation 152 into target region 95 is predetermined by its wavelength. For example, electromagnetic radiation 152 having a wavelength ranging between 0.80-1.3 μm may penetrate deeper into target region 95 compared to electromagnetic radiation 152 having a wavelength ranging between 1.35-155 and/or 1.8-2.1 μm.

FIGS. 5E-5F are high level schematic illustrations of a chisel-like medical treatment device 100, according to some embodiments of the invention. FIG. 5E illustrates a front view of chisel-like medical treatment device 100 (at the top of FIG. 5E) and a distribution 156 of electromagnetic radiation 152 emitted by the device (at the bottom of FIG. 5E). FIG. 5F illustrates an application of chisel-like medical treatment device 100 on a target region 95.

Medical treatment device 100 may be configured to emit electromagnetic radiation 152 from distal end 112 of an optical fiber (e.g., optical fiber 110 and/or optical fiber 130) embedded within the device. In some embodiments, the optical fiber comprises facets 114 at distal end 112 of the fiber. Facets 114 may be configured to distribute electromagnetic radiation 152 in a curtain-like profile generating thereby an optical chisel (e.g., as shown in FIG. 5E). Chisel-like medical treatment device 100 may be used, for example, to cut torn portion 72 of meniscus 70 (e.g., as shown in FIG. 5F).

FIGS. 5G-5H are high level schematic illustrations of a hook-like medical treatment device 100, according to some embodiments of the invention. FIG. 5G illustrates a front view of hook-like medical treatment device 100 (at the top of FIG. 5G) and a distribution 156 of electromagnetic radiation 152 emitted by the device (at the bottom of FIG. 5G).

Hook-like medical treatment device 100 may comprise treatment section 105 (e.g., any of treatment section 105 as shown in FIGS. 3A-3I) positioned at distal end 112 of the device. Treatment section 105 may comprise at least one optical fiber 110 and/or at last one emission region 145. In some embodiments, hook-like medical treatment device comprises optical fiber 130. Curved region 102 of treatment section 105 may comprise three sections 102-1, 102-2, 102-3 having bending radii 101-1, 101-2, 101-3, respectively. In some embodiments, each of bending radii 101-1, 101-2, 101-3 has different value. In various embodiments, bending radius 101-1 is predetermined to transfer an electromagnetic radiation travelling along optical fiber 110 into higher order modes (e.g., as described in detail with respect to FIG. 1D) and/or bending radii 101-2, 101-3 are predetermined to optimize and/or tailor emitted electromagnetic radiation 152 to generate a uniform emission profile 156 (e.g., as shown on the bottom of FIG. 5G).

In various embodiments, hook-like medical treatment device 100 is used to pull and/or cut target region 95, for example torn portion 72 of meniscus 70, as shown in FIG. 5H.

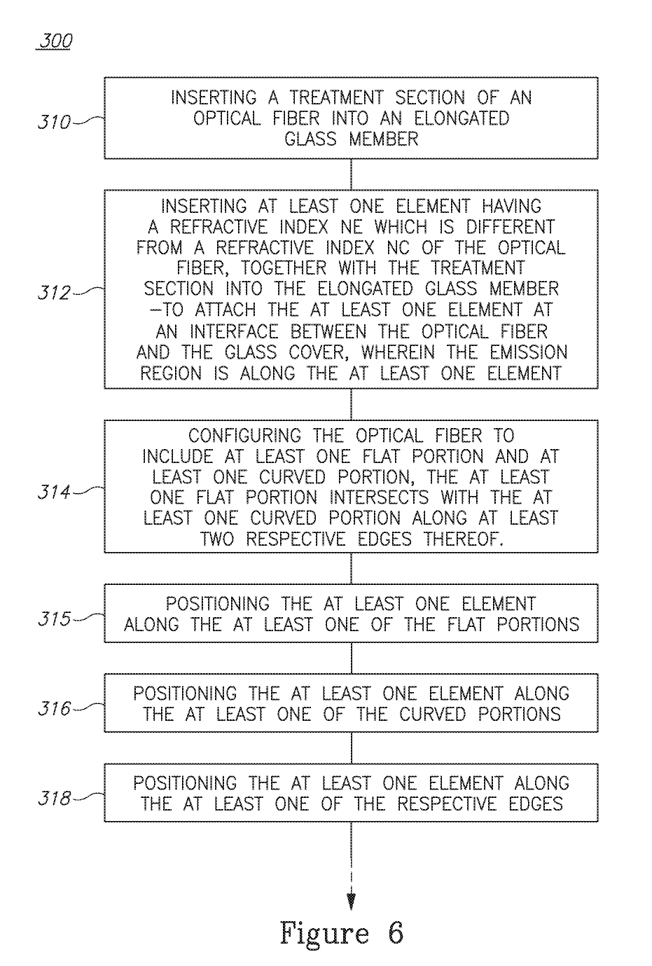

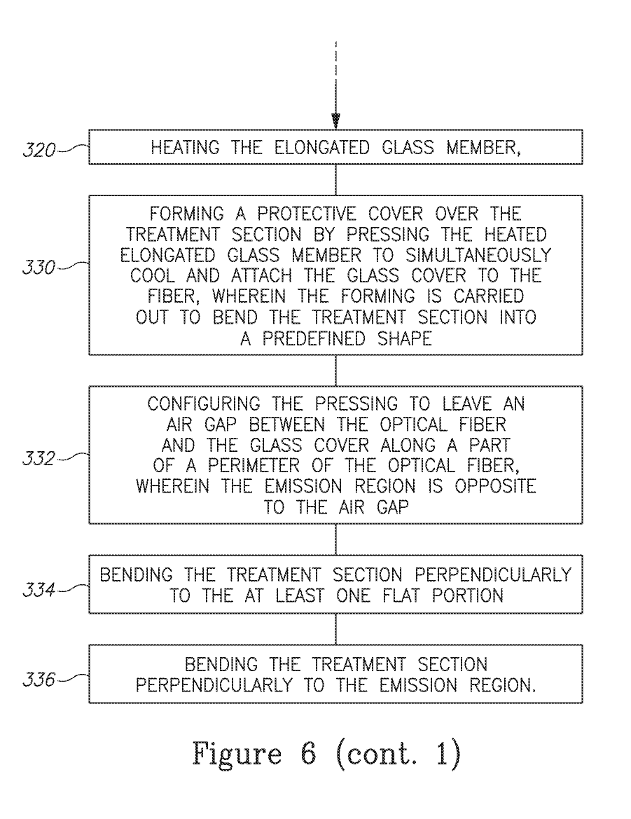

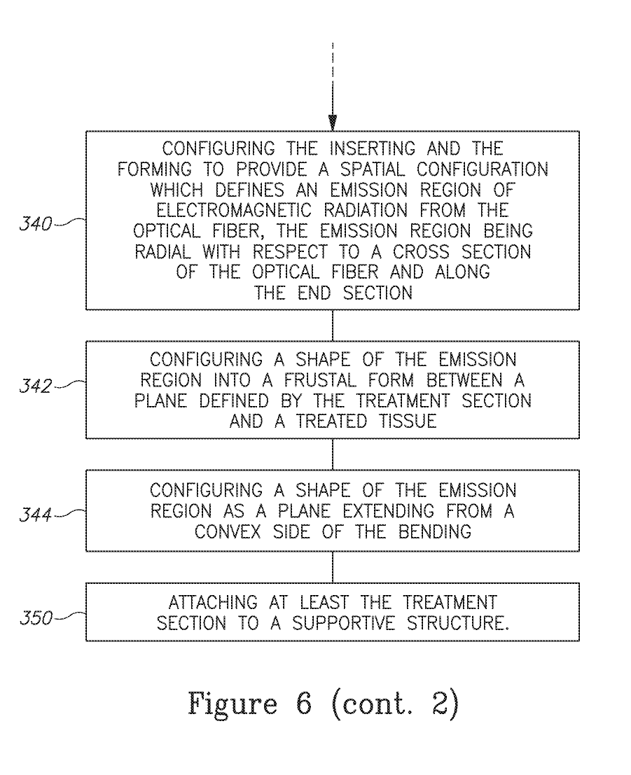

FIG. 6 is a high level schematic flowchart of a method 300 of fusing a glass member 290 to an optical fiber 110 over a treatment section 105 of the optical fiber and bending thereof, according to some embodiments of the invention. Method 300 may be implemented by system 200, which may be configured to implement method 300.

Method 300 may comprise inserting (stage 310) a treatment section (e.g., treatment section 105 as shown in FIG. 1A) of an optical fiber (e.g., optical fiber 110 as shown in FIG. 1A) into an elongated glass member (e.g., glass member 290 as shown in FIG. 2E).

Method 300 may further comprise inserting (stage 312) at least one element having a refractive index n_{E} (e.g., element 140 as shown in FIGS. 3A-3C) which is different from a refractive index n_{C} of the optical fiber, together with the treatment section into the elongated glass member—to attach the at least one element at an interface between the optical fiber and the glass member, wherein the emission region (e.g., emission region 145) is along the at least one element. Method 300 may further comprise configuring (stage 314) the optical fiber to comprise at least one flat portion (e.g., flat portion 116A as shown in FIGS. 3A-3C) and at least one curved portion (e.g., curved portion 116B as shown in FIGS. 3A-3C), the at least one flat portion intersects with the at least one curved portion along at least two respective edges thereof. Method 300 may further comprise positioning (stage 315) the at least one element along the at least one of the flat portions (e.g., as shown in FIG. 3A). Method 300 may further comprise positioning (stage 316) the at least one element along the at least one of the curved portions (e.g., as shown in FIG. 3C). Method 300 may further comprise positioning (stage 318) the at least one element along the at least one of the respective edges (e.g., as shown in FIG. 3B).

Method 300 may comprise heating (stage 320) the elongated glass member (e.g., by heating element 230 as shown in FIGS. 2D-2E).

Method 300 may comprise forming (stage 330) a cover (e.g., cover 120 as shown in FIGS. 1A-1C) over the treatment section by pressing (e.g., by first and/or second bending elements 242, 244 as shown in FIGS. 2A-2E) the heated elongated glass member to simultaneously cool and attach the glass member to the optical fiber, wherein the forming is carried out to bend (e.g., by first and/or second bending elements 242, 244 as shown in FIGS. 2A-2E) the treatment section into a predefined shape (e.g., curved portion 102 as shown in FIGS. 1B-1C).

Method 300 may further comprise configuring (stage 332) the pressing to leave an air gap (e.g., air gap 125 as shown in FIGS. 3D-3E) between the optical fiber and the glass cover along a part of a perimeter of the optical fiber, wherein the emission region is opposite to the air gap (e.g., by controlling positive and negative pressure inside glass member 290). Method 300 may further comprise bending (stage 334) the treatment section perpendicularly to the at least one flat portion (e.g., as shown in FIGS. 3A-3D). Method 300 may further comprise bending (stage 336) the treatment section perpendicularly to the emission region (e.g., as shown in FIGS. 3C, 3J).

Method 300 may comprise configuring (stage 340) the inserting and the forming to provide a spatial configuration (e.g., curved portion 102 as shown in FIGS. 1B-1C) which defines an emission region (e.g., emission region 145) of electromagnetic radiation from the optical fiber, the emission region being radial with respect to a cross section of the optical fiber and along the end section.

Method 300 may further comprise configuring (stage 342) a shape of the emission region into a frustal form between a plane defined by the treatment section and a treated tissue. Method 300 may further comprise configuring (stage 344) a shape of the emission region as a plane extending from a convex side of the bending.

Method 300 may comprise attaching (stage 350) at least the treatment section to a supportive structure (e.g., supportive structure 170 as shown in FIGS. 4H-4K).

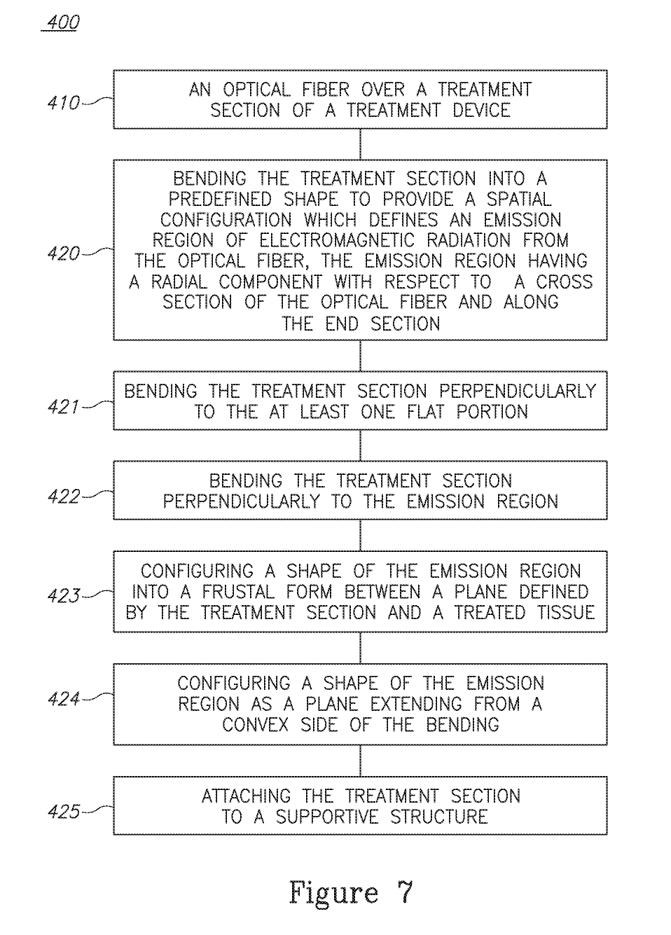

FIG. 7 is a high level schematic flowchart of a method 400 of bending a treatment section 105 of an optical fiber 110, according to some embodiments of the invention. Method 400 may be implemented by system 200, which may be configured to implement method 400.

Method 400 may comprise heating (stage 410) an optical fiber over a treatment section of a medical treatment device. Method 400 may comprise bending (stage 420) treatment section into a predefined shape to provide a spatial configuration which defines an emission region of electromagnetic radiation from the optical fiber, the emission region having a radial component with respect to a cross section of the optical fiber and along the end section.

In some embodiments, method 400 comprises bending (stage 421) the treatment section perpendicularly to the at least one flat portion. In some embodiments, method 400 comprises bending (stage 422) the treatment section perpendicularly to the emission region. In some embodiments, method 400 comprises configuring (stage 423) a shape of the emission region into a frustal form between a plane defined by the treatment section and a treated tissue. In some embodiments, method 400 comprises configuring (stage 424) a shape of the emission region as a plane extending from a convex side of the bending. In some embodiments, method 400 comprises attaching (stage 425) the treatment section to a supportive structure.

In the above description, an embodiment is an example or implementation of the invention. The various appearances of “one embodiment”, “an embodiment”, “certain embodiments” or “some embodiments” do not necessarily all refer to the same embodiments. Although various features of the invention may be described in the context of a single embodiment, the features may also be provided separately or in any suitable combination. Conversely, although the invention may be described herein in the context of separate embodiments for clarity, the invention may also be implemented in a single embodiment. Certain embodiments of the invention may comprise features from different embodiments disclosed above, and certain embodiments may incorporate elements from other embodiments disclosed above. The disclosure of elements of the invention in the context of a specific embodiment is not to be taken as limiting their use in the specific embodiment alone. Furthermore, it is to be understood that the invention may be carried out or practiced in various ways and that the invention may be implemented in certain embodiments other than the ones outlined in the description above.

The invention is not limited to those diagrams or to the corresponding descriptions. For example, flow need not move through each illustrated box or state, or in exactly the same order as illustrated and described. Meanings of technical and scientific terms used herein are to be commonly understood as by one of ordinary skill in the art to which the invention belongs, unless otherwise defined. While the invention has been described with respect to a limited number of embodiments, these should not be construed as limitations on the scope of the invention, but rather as exemplifications of some of the preferred embodiments. Other possible variations, modifications, and applications are also within the scope of the invention. Accordingly, the scope of the invention should not be limited by what has thus far been described, but by the appended claims and their legal equivalents.

1.-21. (canceled)

22. A medical treatment device, the device comprising:
a supportive structure configured to position and to orient at least a portion of the device with respect to a target region;
an optical fiber having a core refractive index n_{C} and a treatment section, wherein at least the treatment section is attached to the supportive structure; and
wherein a spatial configuration of the optical fiber within the treatment section is configured to determine an emission region of electromagnetic radiation from the optical fiber to the target region, the emission region being along the treatment section.

23. The medical treatment device of claim 22, further comprising at least one element embedded within the optical fiber and having a refractive index n_{E} which is different from n_{C}.

24. The medical treatment device of claim 22, further comprising a cover made of deformable and flexible material.

25. The medical treatment device of claim 22, wherein the supportive structure is selected from the group consisting of: a guide-wire, a balloon, a stent, a mesh, a cylinder, a nitinol tube, a catheter tube and any combination thereof.

26. The medical treatment device of claim 24, further comprising at least one element embedded within the optical fiber and having a refractive index n_{E} which is different from n_{C}, wherein the emission region is further determined based on optical properties of at least one of: the optical fiber, the cover, the at least one element or any combination thereof.

27. The medical treatment device of claim 22, wherein the spatial configuration of the optical fiber comprises bending of the optical fiber beyond a bending threshold.

28.-35. (canceled)

36. The medical treatment device of claim 27, wherein the bending is configured to shape the emission region as a plane extending from a convex side of the bending.

37. (canceled)

38. The medical treatment device of claim 22, configured to generate conduction blocks in at least one of: a pulmonary vein, a left atrium, an intersection region of the pulmonary vein and the left atrium, a left ventricle and a right ventricle.

39.-44. (canceled)

45. A medical treatment device comprising:
a supportive structure;
an optical fiber having a treatment section, wherein at least the treatment section is attached to the supportive structure, the treatment section having at least one emission region configured to emit electromagnetic radiation upon bending of the treatment section beyond a bending threshold.

46. The medical treatment device of claim 45, wherein the emission region is further configured to emit the electromagnetic radio from a convex side of the treatment section.

47. (canceled)

48. The medical treatment device of claim 45, wherein the bending of the treatment section is stationary.

49. The medical treatment device of claim 45, wherein the bending of the treatment section is dynamic.

50. The medical treatment device of claim 45, wherein the emission region is further configured to emit the electromagnetic radiation from a concave side of the treatment section.

51. The medical treatment device of claim 50, wherein the treatment section further comprises a first bent section, a second bent section and a third bent section, wherein the first bent section is positioned at a proximal end of the treatment section, the third bent section is positioned at a distal end of the treatment section and the second bent section is positioned between the first and the third bent section.

52. (canceled)

53. The medical treatment device of claim 51, wherein the first bent section has a first bending radius, the second bent section has a second bending radius and the third bent section has a third bending radius, and wherein the first bending radius is configured to transfer the electromagnetic radiation travelling along the optical fiber into higher order modes and wherein the second and the third bending radii are configured to tailor the emitted electromagnetic radiation to generate a uniform emission profile.

54-79. (canceled)

80. A method for medical treatment comprising:
heating an optical fiber over a treatment section of a treatment device;
bending the treatment section into a predefined shape to provide a spatial configuration which defines an emission region of electromagnetic radiation from the optical fiber, the emission region having a radial component with respect to a cross section of the optical fiber and along the end section.

81. (canceled)

82. The method of claim 80, further comprising bending the treatment section in a plane of the emission region.

83. (canceled)

84. The method of claim 82, further comprising configuring a shape of the emission region as a plane extending from a convex side of the bending.

85. The method of claim 80, further comprising attaching the treatment section to a supportive structure.

86. The medical treatment device of claim 22, wherein the supportive structure is selected from the group consisting of forceps, scissors and tweezers.
